SEQUENCE LISTING (23 sequences)

SEQ ID NO: 1 - protein, 19 aa, Bos taurus
MENKWFLSMVRDDFKGGKI

SEQ ID NO: 2 - DNA, 17 bp, Bos taurus
gcccggcttccacttca

SEQ ID NO: 3 - DNA, 21 bp, Bos taurus
ggcctcgtacagctcgaagta

SEQ ID NO: 4 - DNA, 23 bp, Bos taurus
gcaaaggaaaggaaaatcaacaa

SEQ ID NO: 5 - DNA, 18 bp, Bos taurus
gggctctgggacgcttct

SEQ ID NO: 6 - DNA, 19 bp, Bos taurus
tgttgcagcgaacgacaag

SEQ ID NO: 7 - DNA, 21 bp, Bos taurus
ccgcaagtgtcctaaatttgg

SEQ ID NO: 8 - DNA, 22 bp, Bos taurus
ctggcagctaaatctcgatgaa

SEQ ID NO: 9 - DNA, 23 bp, Bos taurus
gacctggtgtcgtgtacctacca

SEQ ID NO: 10 - DNA, 20 bp, Bos taurus
gccatcaatgaccccttcat

SEQ ID NO: 11 - DNA, 17 bp, Bos taurus
tgccgtgggtggaatca

SEQ ID NO: 12 - DNA, 20 bp, Bos taurus
tccggcagggaggagcaagt

SEQ ID NO: 13 - DNA, 22 bp, Bos taurus
tgctgagatctatcagtttgag

SEQ ID NO: 14 - DNA, 20 bp, Bos taurus
cgtgtccttgcaaacgtcat

SEQ ID NO: 15 - DNA, 22 bp, Bos taurus
ctgtctctcctcttccctcctc

SEQ ID NO: 16 - DNA, 20 bp, Bos taurus
ccaccctgcagcaaattagc

SEQ ID NO: 17 - DNA, 20 bp, Bos taurus
ccacactcggaccacgtctt

SEQ ID NO: 18 - DNA, 29 bp, Bos taurus
ggttgacatcgttggtaatttataatagc

SEQ ID NO: 19 - DNA, 26 bp, Bos taurus
cacagtaatttcatgttggtttttca

SEQ ID NO: 20 - DNA, 20 bp, Bos taurus
ccacactcggaccacgtctt

SEQ ID NO: 21 - DNA, 19 bp, Bos taurus
agccagcgcagatcatgtt

SEQ ID NO: 22 - DNA, 22 bp, Bos taurus
ggagtagtcatgagggcgagaa

SEQ ID NO: 23 - DNA, 21 bp, Bos taurus
ttccgctgctttgagaactgt

(19) United States
(12) Patent Application Publication
(10) Pub. No.: US 2008/0175827 A1
(43) Pub. Date: Jul. 24, 2008

(54) PHOSPHOLIPASE C ZETA MEDIATED OOCYTE ACTIVATION, COMPOSITIONS FOR USE THEREIN, AND ASSAYS FOR DETECTING AND IDENTIFYING AGENTS FOR TREATING MALE INFERTILITY

(75) Inventors: Rafael A. Fissore, Amherst, MA (US); Pablo Visconti, Amherst, MA (US); Manabu Kurokawa, Durham, NC (US); Sook-Young Yoon, Amherst, MA (US); Daniel R. Grow, Long Meadow, MA (US); Teru J. Jellerette, Shutesbury, MA (US); Jose Bernardo Cibelli, East Lansing, MI (US); Pablo Juan Ross, Bath, MI (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignees: Michigan State University, East Lansing, MI (US); University of Massachussetts, Boston, MA (US); Baystate Health, Inc., Springfield, MA (US)

(21) Appl. No.: 11/942,406

(22) Filed: Nov. 19, 2007

Related U.S. Application Data
(60) Provisional application No. 60/866,319, filed on Nov. 17, 2006.

(51) Int. Cl. A61K 48/00; C12N 5/00; C12N 15/00; C12Q 1/68; A01N 1/02; C12Q 1/42

(52) U.S. Cl. 424/93.21; 435/377; 435/455; 435/2; 435/21; 435/6

(57) ABSTRACT

The invention relates to the use of phospholipase C zeta in inducing or promoting the activation of oocytes, preferably human oocytes, nuclear transfer embryos, parthenogenic embryos, cross species embryos and during in vitro fertilization.

The invention further relates to methods of detecting levels of expression of phospholipase c zeta in sperm as a means of detecting male infertility.

The invention also relates to the use of phospholipase C zeta to treat male infertility. Still further the invention relates to the recombinant cells that are engineered to express phospholipase C zeta, preferably under inducible conditions.

Also, the invention relates to antibodies specific to phospholipase C zeta, preferably human phospholipase C zeta.

This application is a non-provisional of provisional application Ser. No. 60/866,319 filed Nov. 17, 2006, hereby incorporated by reference thereto as if fully set forth herein.

### FIELD OF THE INVENTION

The invention relates to the use of phospholipase C zeta in inducing or promoting the activation of oocytes, preferably human oocytes, nuclear transfer cells, parthenogenic cells, and during in vitro fertilization.

The invention further relates to methods of detecting levels of expression of phospholipase c zeta in sperm as a means of detecting male infertility.

The invention also relates to the use of phospholipase C zeta to treat male infertility. Still further the invention relates to the recombinant cells that are engineered to express phospholipase C zeta, preferably under inducible conditions.

Also, the invention relates to antibodies specific to phospholipase C zeta, preferably human phospholipase C zeta.

### BACKGROUND OF THE INVENTION

Ovulated mammalian oocytes are arrested at the metaphase II (MII) stage of meiosis and only complete meiosis after fertilization. Sperm is responsible for releasing the oocyte from its meiotic arrest and also for inducing other events that are collectively referred to as oocyte activation and include cortical granule exocytosis, reinitiation of meiosis, extrusion of the second polar body, formation of pronuclei, and recruitment of mRNA (See Ducibella, T., et al., 2002. Egg-to-embryo transition is driven by differential responses to Ca(2+) oscillation number. Dev Biol. 250, 280-91; See Schultz, R. M., Kopf, G. S., 1995. Molecular basis of mammalian egg activation. Curr Top Dev Biol. 30, 21-62). In all mammalian species studied so far, activation of oocytes is triggered by repetitive rises in intracellular free-Ca²⁺ concentration ([Ca²⁺]ᵢ) (See Miyazaki, S., et al., 1993. Essential role of the inositol 1,4,5-trisphosphate receptor/Ca2+ release channel in Ca2+ waves and Ca2+ oscillations at fertilization of mammalian eggs. Dev Biol. 158, 62-78), a sufficient and indispensable event (See Jones, K. T., 1998. Ca2+ oscillations in the activation of the egg and development of the embryo in mammals. Int J Dev Biol. 42, 1-10). The rises are also referred to as transients, fluctuations, responses, and other similar terms.

The [Ca²⁺]ᵢ rises are generated by release of Ca²⁺ from intracellular stores mediated by the inositol 1,4,5-triphosphate (IP₃) signaling pathway (See Stith, B. J., et al., 1994. Sperm increase inositol 1,4,5-trisphosphate mass in Xenopus laevis eggs preinjected with calcium buffers or heparin. Dev Biol. 165, 206-15; See Turner, P. R., et al., 1984. Fertilization increases the polyphosphoinositide content of sea urchin eggs. Nature. 310, 414-5).

It is hypothesized that upon fusion with the oocyte the sperm introduces a protein factor responsible for inducing Ca²⁺ release by production of IP₃. One attractive candidate for the factor the sperm delivers into the oocyte is phospholipase C zeta (PLC zeta) (See Malcuit, C., et al., 2006. Calcium oscillations and mammalian egg activation. J Cell Physiol. 206, 565-73). This PLC variant is sperm specific (See Saunders, C. M., et al., 2002. PLC zeta: a sperm-specific trigger of Ca(2+) oscillations in eggs and embryo development. Development. 129, 3533-44) and, when introduced into mouse oocytes, induces sperm-like [Ca²⁺]ᵢ oscillations (See Fujimoto, S., et al., 2004. Mammalian phospholipase Czeta induces oocyte activation from the sperm perinuclear matrix. Dev Biol. 274, 370-83). When cRNA coding for PLC zeta is introduced into mouse (See Saunders, C. M., et al., 2002. PLC zeta: a sperm-specific trigger of Ca(2+) oscillations in eggs and embryo development. Development. 129, 3533-44), human (See Rogers, N. T., et al., 2004. Phospholipase Czeta causes Ca2+ oscillations and parthenogenetic activation of human oocytes. Reproduction. 128, 697-702), and pig (See Yoneda, A., et al., 2006. Molecular cloning, testicular postnatal expression, and oocyte-activating potential of porcine phospholipase Czeta. Reproduction. 132, 393-401) matured oocytes, it induces [Ca²⁺]ᵢ oscillations and oocyte activation. In mouse sperm, PLC zeta localizes to the postacrosomal region (See Fujimoto, S., et al., 2004. Mammalian phospholipase Czeta induces oocyte activation from the sperm perinuclear matrix. Dev Biol. 274, 370-83), the area thought to first interact with the oocyte membrane (See Sutovsky, P., et al., 2003. Interactions of sperm perinuclear theca with the oocyte: implications for oocyte activation, anti-polyspermy defense, and assisted reproduction. Microsc Res Tech. 61, 362-78). Functional studies using RNAi to reduce the level of PLC zeta in sperm showed that [Ca²⁺]ᵢ oscillations were reduced after intracytoplasmic sperm injection (ICSI), and a lower number of progeny was obtained after natural mating (See Knott, J. G., et al., 2005. Transgenic RNA interference reveals role for mouse sperm phospholipase Czeta in triggering Ca2+ oscillations during fertilization. Biol Reprod. 72, 992-6). Finally, in fractionation studies, the presence of immunoreactive PLC zeta correlated with the ability of the fractions to induce oocyte activation (See Fujimoto, S., et al., 2004. Mammalian phospholipase Czeta induces oocyte activation from the sperm perinuclear matrix. Dev Biol. 274, 370-83), and immunodepletion of PLC zeta from sperm extracts suppressed its [Ca²⁺]ᵢ oscillation-inducing ability (See Saunders, C. M., et al., 2002. PLC zeta: a sperm-specific trigger of Ca(2+) oscillations in eggs and embryo development. Development. 129, 3533-44). Altogether, this evidence is consistent with the results presented herein indicating that PLC zeta delivered into the oocyte upon sperm-oocyte fusion is the factor responsible for oocyte activation.

PLC zeta, like other PLCs, catalyzes the hydrolysis of phosphatidyl 4,5-bisphosphate (PIP₂), producing IP₃ and 1,2-diacylglycerol (DAG). The elevation of IP₃ concentration is responsible for inducing Ca²⁺ release from the endoplasmic reticulum (ER) by binding to the IP₃R. The mechanism responsible for maintaining [Ca²⁺]ᵢ oscillations for long periods is not clear; however, IP₃R-1 is believed to play an important role in controlling the duration of [Ca²⁺]ᵢ oscillations in mammalian oocytes (See Lee, B., et al., 2006b. Regulation of fertilization-initiated [Ca2+]i oscillations in mammalian eggs: a multi-pronged approach. Semin Cell Dev Biol. 17, 274-84; See Malcuit, C., et al., 2006. Calcium oscillations and mammalian egg activation. J Cell Physiol. 206, 565-73). Following fertilization, IP3R-1 is degraded to about 50 percent the amount of the receptor present in the MII stage oocyte (See Brind, S., et al., 2000. Inositol 1,4,5-trisphosphate receptors are downregulated in mouse oocytes in response to sperm or adenophostin A but not to increases in intracellular Ca(2+) or egg activation. Dev Biol. 223, 251-65; See Jellerette, T., et al., 2000. Down-regulation of the inositol 1,4,5-trisphosphate receptor in mouse eggs following fertilization or parthenogenetic activation. Dev Biol. 223, 238-50). IP₃R-1 downregulation is believed to contribute to the decreased responsiveness to IP₃ observed after fertilization (See Jellerette, T., et al., 2004. Cell cycle-coupled [Ca(2+)](i) oscillations in mouse zygotes and function of the inositol 1,4,5-trisphosphate receptor-1. Dev Biol. 274, 94-109). Moreover, post-translational modifications of IP₃R-1 by cell-cycle-dependent kinases may also play an important role in reducing IP₃R-1 activity (See Lee, B., et al., 2006a. Phosphorylation of IP3R1 and the regulation of [Ca2+]i responses at fertilization: a role for the MAP kinase pathway. Development. 133, 4355-65). While PLC zeta clearly triggers [Ca²⁺]ᵢ oscillations in the oocyte, its role in regulating IP₃R-1 has not been previously reported.

In addition the potential role of phospholipase C zeta in the activation of oocytes has previously been suggested in the following references, (See Cox, L. J., et al., 2002. Sperm phospholipase Czeta from humans and cynomolgus monkeys triggers Ca2+ oscillations, activation and development of mouse oocytes. Reproduction. 124, 611-23; Momikos et al., J Biol. Chem. 280(35):31011-8 (2005); Fujimoto et al., Dev. Biol. 274(2):370-383 (2004); Dominguez et al., Mol. Cell Biol. 12(9):3776-83 (1992); Dominguez et al., Mol Cell Biol. 13(2):1290-5 (1993); and Berra et al., Cell 74(3):555-63 (1993)).

Generation of live offspring after somatic cell nuclear transfer (SCNT) has been successfully achieved in several mammalian species (See, Cibelli J. Developmental biology. A decade of cloning mystique. Science 2007; 316: 990-992); however, the overall efficiency of the technology remains extremely low (See, Wilmut I, Beaujean N, de Sousa P A, Dinnyes A, King T J, Paterson L A, Wells D N, Young L E. Somatic cell nuclear transfer. Nature 2002; 419: 583-587). Failure to correctly reprogram the somatic cell genome following transfer into the oocyte cytoplasm is hypothesized to be a major cause of developmental abnormalities (See, Hochedlinger K, Jaenisch R. Nuclear reprogramming and pluripotency. Nature 2006; 441: 1061-1067.; see, Shi W, Zakhartchenko V, Wolf E. Epigenetic reprogramming in mammalian nuclear transfer. Differentiation 2003; 71: 91-113). Nuclear reprogramming, also known as activation, is the process by which a specialized nucleus re-acquires developmental potential, adopting the role of a zygotic nucleus. This process involves the silencing of somatic-specific genes and activation of essential embryonic genes (See, Latham K E. Early and delayed aspects of nuclear reprogramming during cloning. Biol Cell 2005; 97: 119-132). Although the process of nuclear reprogramming is not fully understood, it is becoming more and more evident that epigenetic modifications of chromatin (e.g. methylation and/or acetylation of DNA and histones) is fundamental for regulation of gene expression (See, Rideout W M, III, Eggan K, Jaenisch R. Nuclear Cloning and Epigenetic Reprogramming of the Genome. Science 2001; 293: 1093-1098.; see, Reik W, Dean W, Walter J. Epigenetic Reprogramming in Mammalian Development. Science 2001; 293: 1089-1093).

In most mammalian species, oocytes are ovulated at the MII stage of meiosis and remain arrested until fertilized by sperm. Initiation of development is triggered by a series of long lasting intracellular free-calcium ([Ca²⁺]ᵢ) oscillations. Several pieces of evidence are consistent with the hypothesis that the sperm, upon fusion with the oocyte, delivers a sperm-specific isoform of phospholipase C(PLCZ) (See, Saunders C M, Larman M G, Parrington J, Cox L J, Royse J, Blayney L M, Swann K, Lai F A. PLC zeta: a sperm-specific trigger of Ca(2+) oscillations in eggs and embryo development. Development 2002; 129: 3533-3544; see, Knott J G, Kurokawa M, Fissore R A, Schultz R M, Williams C J. Transgenic RNA interference reveals role for mouse sperm phospholipase Czeta in triggering Ca2+ oscillations during fertilization. Biol Reprod 2005; 72: 992-996; see, Malcuit C, Kurokawa M, Fissore R A. Calcium oscillations and mammalian egg activation. J Cell Physiol 2006; 206: 565-573; see, Kurokawa M, Sato K, Fissore R A. Mammalian fertilization: from sperm factor to phospholipase Czeta. Biol Cell 2004; 96: 37-45; see, Swann K, Saunders C M, Rogers N T, Lai F A. PLCzeta(zeta): a sperm protein that triggers Ca2+ oscillations and egg activation in mammals. Semin Cell Dev Biol 2006; 17: 264-273; see, Swann K, Larman M G, Saunders C M, Lai F A. The cytosolic sperm factor that triggers Ca2+ oscillations and egg activation in mammals is a novel phospholipase C: PLCzeta. Reproduction 2004; 127: 431-439). PLCZ has the ability to function at basal Ca²⁺ concentrations, and thus, upon entering the oocyte's cytoplasm, induces hydrolysis of phosphatidylinositol-4,5-bisphosphate (PIP₂) generating 1,2-diacylglycerol (DAG) and inositol-1,4,5-tri-phosphate (IP₃). In turn, IP₃ binds to its receptor (IP₃R), located on the endoplasmic reticulum (ER) membrane and induces a conformational change that allows the free diffusion of Ca²⁺ into the oocyte's cytoplasm. By a yet uncharacterized mechanism, Ca²⁺ release and uptake are repeated, generating what is described as [Ca²⁺]ᵢ oscillations. The pattern of [Ca²⁺]ᵢ oscillations is species-specific and observed over a relatively long period of time (3-4 hours in mice; 16-18 hours in cattle).

The importance of the [Ca²⁺]ᵢ oscillatory pattern has been well-studied in mice and rabbits (See. Ozil J P, Huneau D. Activation of rabbit oocytes: the impact of the Ca2+ signal regime on development. Development 2001; 128: 917-928; see, Ducibella T, Schultz R M, Ozil J P. Role of calcium signals in early development. Semin Cell Dev Biol 2006; 17: 324-332). It has been shown that alterations in calcium signaling not only affect the early events of embryonic development see, Ducibella T, Huneau D, Angelichio E, Xu Z, Schultz R M, Kopf G S, Fissore R, Madoux S, Ozil J P. Egg-to-embryo transition is driven by differential responses to Ca(2+) oscillation number. Dev Biol 2002; 250: 280-291; see, Ozil J P, Markoulaki S, Toth S, Matson S, Banrezes B, Knott J G, Schultz R M, Huneau D, Ducibella T. Egg activation events are regulated by the duration of a sustained [Ca2+]cyt signal in the mouse. Dev Biol 2005; 282: 39-54), but also gene expression in 8-cell (See, Rogers N T, Halet G, Piao Y, Carroll J, Ko M S, Swann K. The absence of a Ca(2+) signal during mouse egg activation can affect parthenogenetic preimplantation development, gene expression patterns, and blastocyst quality. Reproduction 2006; 132: 45-57) and blastocyst stage embryos (See, Ozil J P, Banrezes B, Toth S, Pan H, Schultz R M. Ca2+ oscillatory pattern in fertilized mouse eggs affects gene expression and development to term. Dev Biol 2006; 300: 534-544), implantation (See, Ozil J P, Huneau D. Activation of rabbit oocytes: the impact of the Ca2+ signal regime on development. Development 2001; 128: 917-928; see, Ozil J P, Banrezes B, Toth S, Pan H, Schultz R M. Ca2+ oscillatory pattern in fertilized mouse eggs affects gene expression and development to term. Dev Biol 2006; 300: 534-544), and even development to term (See, Ozil J P, Banrezes B, Toth S, Pan H, Schultz R M. Ca2+ oscillatory pattern in fertilized mouse eggs affects gene expression and development to term. Dev Biol 2006; 300: 534-544). It is possible that the long-lasting effect of [Ca²⁺]ᵢ oscillations is mediated by alterations in chromatin structure and reprogramming of gene expression that occur after fertilization (See. Ozil J P, Huneau D. Activation of rabbit oocytes: the impact of the Ca2+ signal regime on development.

Development 2001; 128: 917-928; see, Rogers N T, Halet G, Piao Y, Carroll J, Ko M S, Swann K. The absence of a Ca(2+) signal during mouse egg activation can affect parthenogenetic preimplantation development, gene expression patterns, and blastocyst quality. Reproduction 2006; 132: 45-57; see, Ozil J P, Banrezes B, Toth S, Pan H, Schultz R M. Ca2+ oscillatory pattern in fertilized mouse eggs affects gene expression and development to term. Dev Biol 2006; 300: 534-544). These observations suggest that improper oocyte activation may affect the level of nuclear reprogramming following SCNT.

Most activation protocols commonly used during SCNT rely on chemicals that not only induce a non-physiological Ca²⁺-transient pattern, but also affect other cellular processes, with possible negative consequences for embryonic development (See, Alberio R, Zakhartchenko V, Motlik J, Wolf E. Mammalian oocyte activation: lessons from the sperm and implications for nuclear transfer. Int J Dev Biol 2001; 45: 797-809; see, Alberio R, Brero A, Motlik J, Cremer T, Wolf E, Zakhartchenko V. Remodeling of donor nuclei, DNA-synthesis, and ploidy of bovine cumulus cell nuclear transfer embryos: effect of activation protocol. Mol Reprod Dev 2001; 59: 371-379). For example, cycloheximide use during activation of bovine SCNT embryos has been associated with delayed DNA synthesis (See, Alberio R, Brero A, Motlik J, Cremer T, Wolf E, Zakhartchenko V. Remodeling of donor nuclei, DNA-synthesis, and ploidy of bovine cumulus cell nuclear transfer embryos: effect of activation protocol. Mol Reprod Dev 2001; 59: 371-379), and the use of 6-DMAP as the activating agent often results in a high proportion of aneuploid embryos (See, Bhak J S, Lee S L, Ock S A, Mohana Kumar B, Choe S Y, Rho G J. Developmental rate and ploidy of embryos produced by nuclear transfer with different activation treatments in cattle. Anim Reprod Sci 2006; 92: 37-49; see, Van De Velde A, Liu L, Bols P E, Ysebaert M T, Yang X. Cell allocation and chromosomal complement of parthenogenetic and IVF bovine embryos. Mol Reprod Dev 1999; 54: 57-62; see, Winger Q A, De La Fuente R, King W A, Armstrong D T, Watson A J. Bovine parthenogenesis is characterized by abnormal chromosomal complements: implications for maternal and paternal co-dependence during early bovine development. Dev Genet 1997; 21: 160-166).

### DETAILED DESCRIPTION OF THE FIGURES

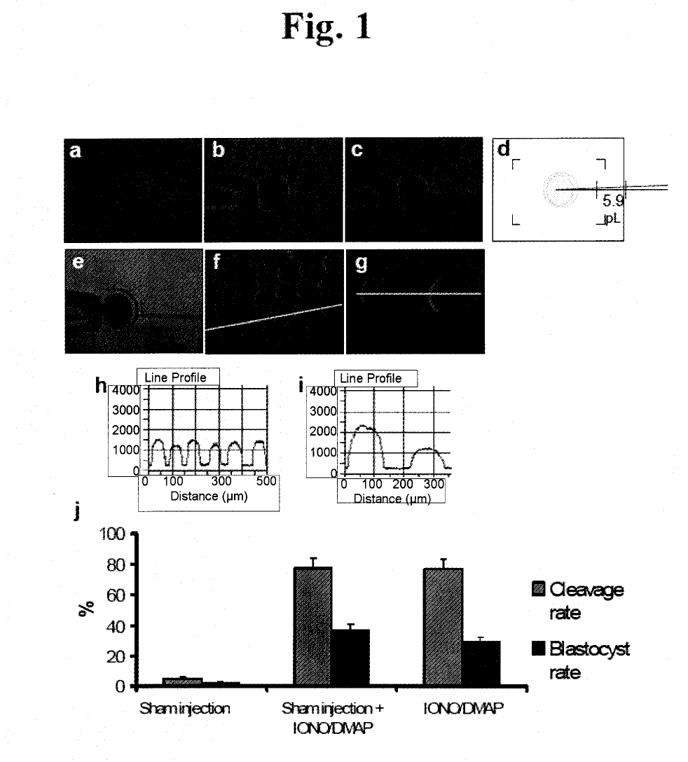

FIG. 1: Validation of intracytoplasmic injection technique. a, b, c: Sequence of injection. a) Pipette loaded with Texas Red dextran just before injection. b) Pipette advanced into the oocyte; cytoplasm is aspirated to break the plasma membrane. c) Aspirated cytoplasm and Texas Red dextran are injected into the oocyte. d) Schematic representation of the microscope reticulum used as guide to control the injected volume. The oocyte is represented in yellow and the pipette in blue. The red lines indicate the volume introduced into the oocyte which, calculated measuring the pipette internal diameters at both ends, is 5.9 pL. e) An oil drop of the same size as the injected volume is shown next to an oocyte. f) Oocytes injected using Texas Red dextran. g) From left to right, oocyte injected 2× and 1× the normal volume of Texas Red dextran. h) Fluorescent intensity profile of the line shown in f. i) Fluorescent intensity profile of the line shown in g. j) Developmental rates of injected and uninjected bovine oocytes after activation using ionomycin/DMAP.

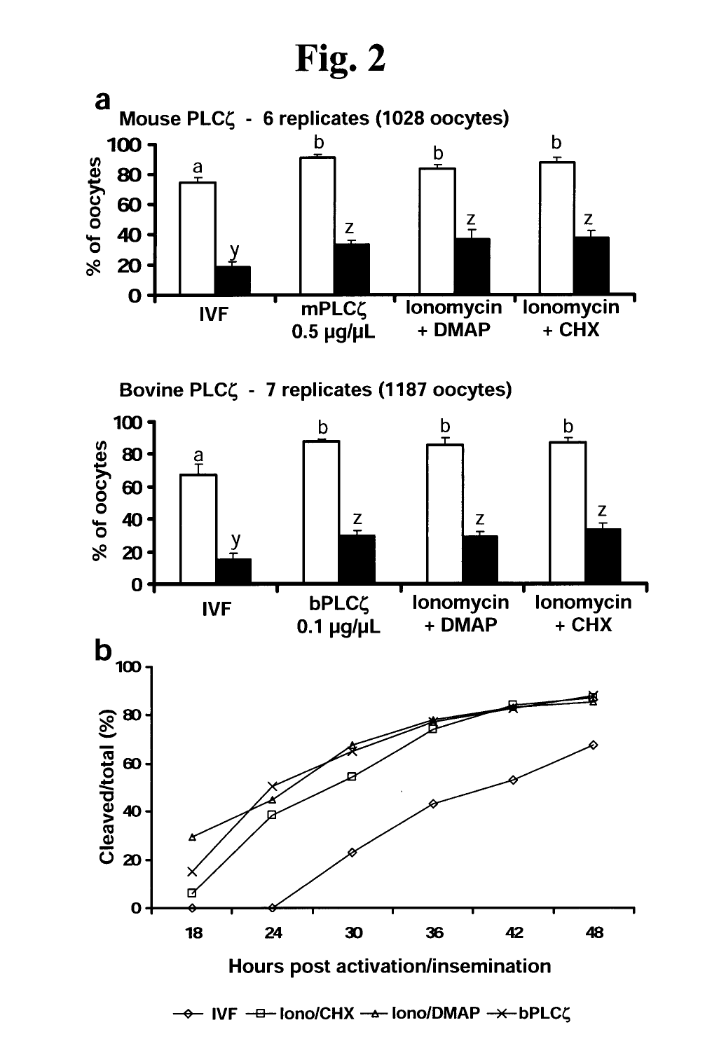

FIG. 2: PLC zeta cRNA injection induces bovine parthenogenetic development at similar rates to common chemical activation protocols. a) Mean cleavage (open bars) and blastocyst (black bars) rates after IVF or parthenogenetic activation using different methods. Error bar represents SEM. a, b or y, z: different superscripts represent P<0.05. b) Cleavage of embryos after IVF or parthenogenetic activation using different methods. Iono=ionomycin; CHX=cycloheximide.

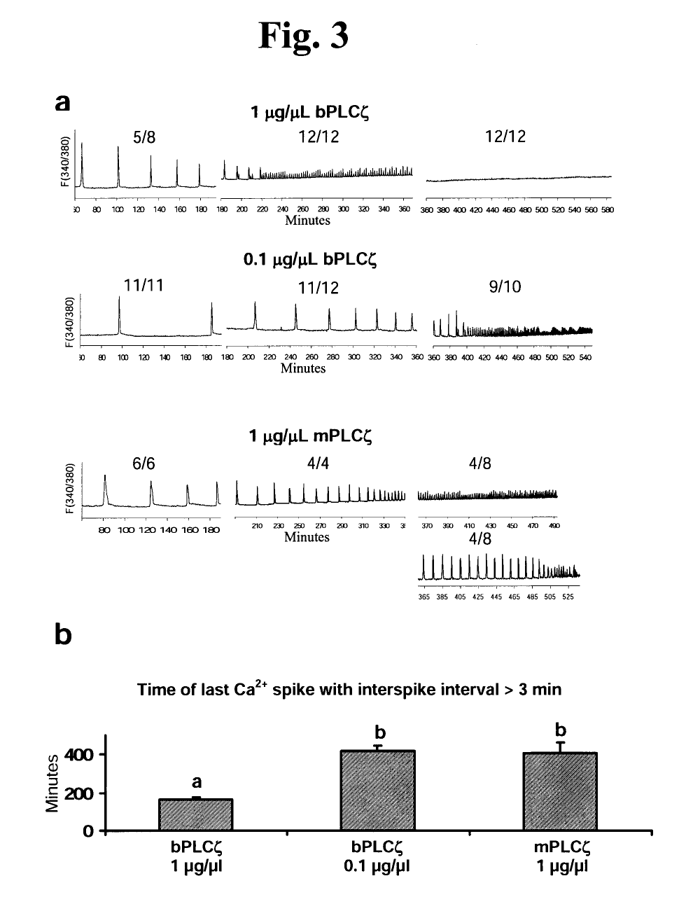

FIG. 3: PLC zeta cRNA injection induces sperm-like calcium oscillations. a) Representative [Ca²⁺]ᵢ profiles. The fluorescence intensity ratio at 340/380 nm is plotted over time (minutes after PLC zeta injection). The number above each graph represents the proportion of oocytes analyzed that displayed a similar pattern to that shown. b) Minutes after injection at which the [Ca²⁺]ᵢ pattern changed from interspike intervals of >3 minutes to <3 minutes for each treatment. Data represented as mean ±SEM. Different letters indicate P<0.06.

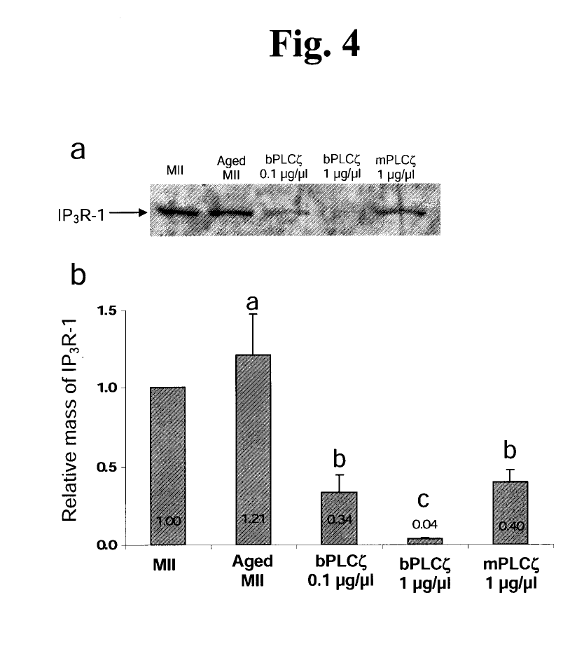

FIG. 4: PLC zeta cRNA injection induces IP3R-1 downregulation. a) Immunoblot. Five bovine oocytes were used per lane; samples were collected 12 hours after cRNA injection. MII oocytes were collected at the time of cRNA injection. Aged MII are noninjected oocytes that were left in culture the same amount of time as the injected ones. bPLC zeta=bovine PLC zeta; mPLC zeta=mouse PLC zeta b) Quantification of IP3R-1 mass relative to the levels observed in MII oocytes. The number in the bars indicates relative IP₃R-1 mass. Data represented as mean ±SEM of two replications. Different letters indicate P<0.06.

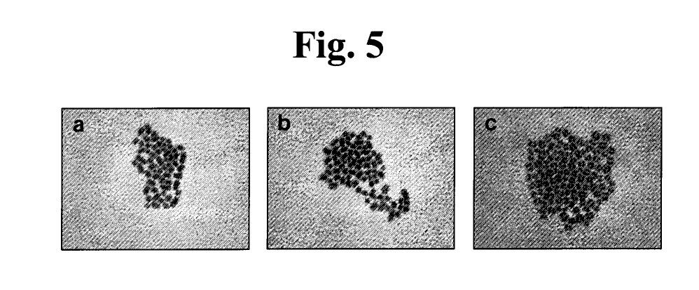

FIG. 5: Representative chromosomal spreads from 8-cell stage bovine embryos (1000×). a) Diploid cell, b) triploid cell, and c) tetraploid cell.

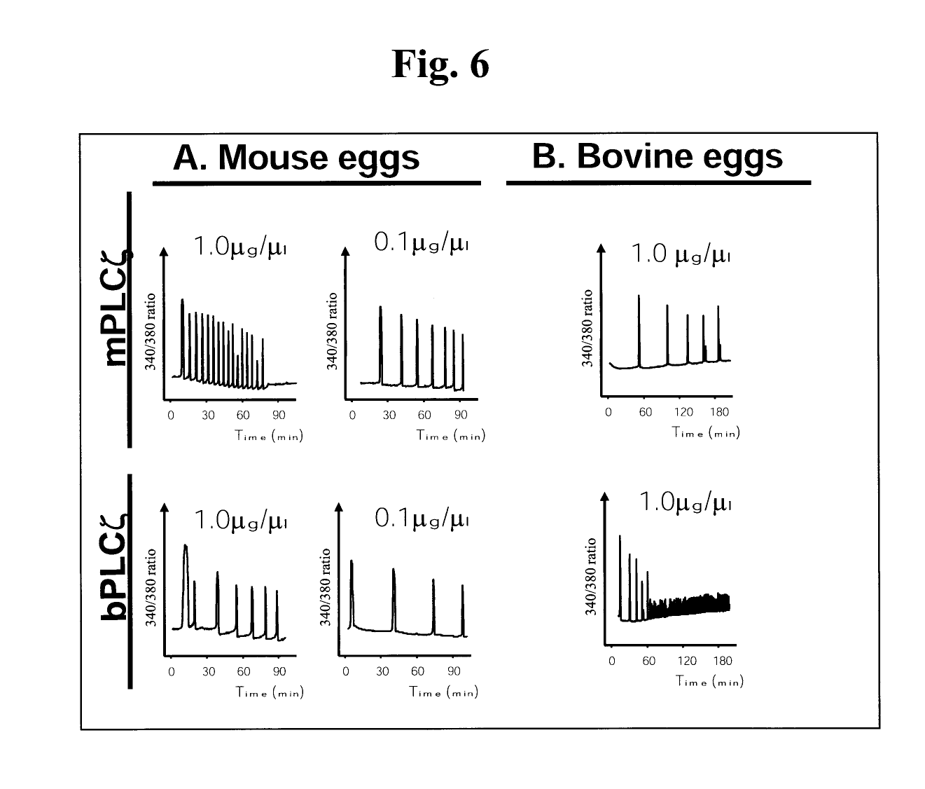

FIG. 6: PLC zeta initiates species-specific [Ca2+]ᵢ oscillations in mammalian eggs. Representative traces show Ca2+ oscillations following introduction of PLZ zeta cRNA into eggs. (A) Mouse eggs exhibit more frequent Ca2+ oscillations in response to mouse PLC zeta cRNA than to the same concentrations of bovine PLZ zeta cRNA. (B) Bovine eggs exhibit more frequent Ca2+ oscillations in response to bovine PLC zeta cRNA than to the same concentration of mouse PLZ zeta cRNA.

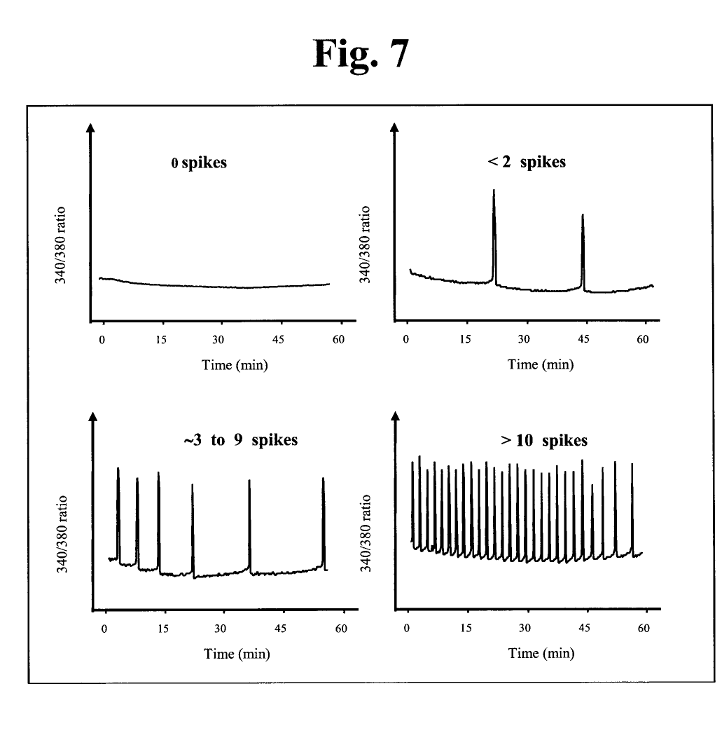

FIG. 7: Injection of sperm from low fertility patients into mouse eggs initiates highly different [Ca2+]i responses. Representative traces of Ca2+ oscillations observed after introduction of sperm from patients exhibiting low fertility or controls having normal fertility.

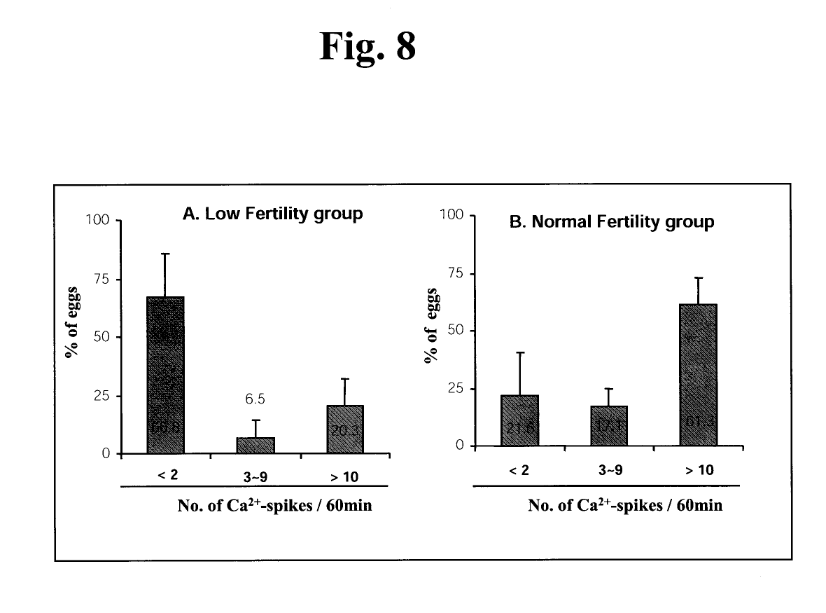

FIG. 8: [Ca2+]i patterns in mouse eggs by human sperm vary according to fertility status. Calcium oscillations observed in 60 minutes following injection of sperm from (A) patients having low fertility or (B) controls having normal fertility are shown as a percentage of samples tested having two or fewer, three to nine, or ten or more Ca2+ in 60 minutes following the injection.

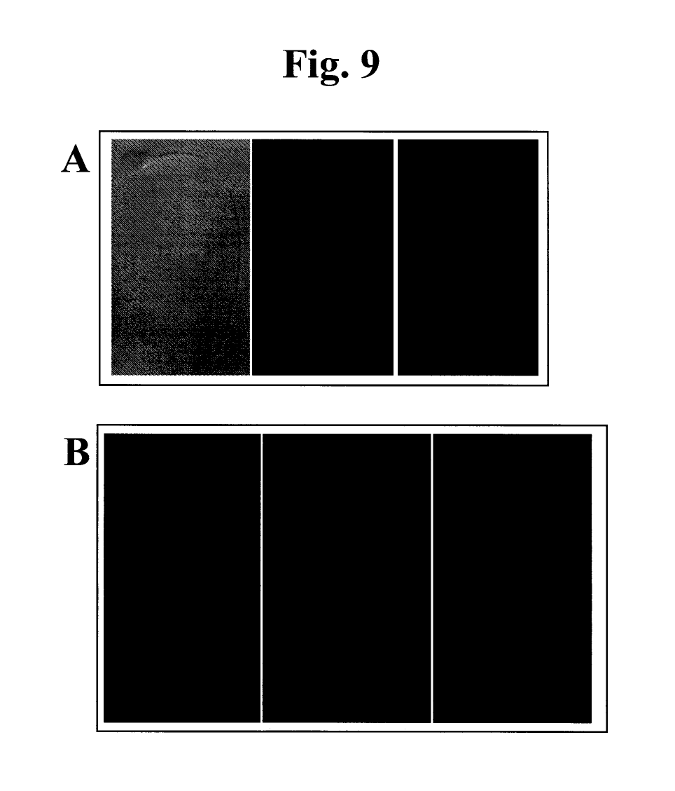

FIG. 9: Localization of PLC zeta in human sperm. (A) Human sperm were immunostained with a negative control primary antibody, or negative control antibody. First panel, DIC microscopy. Second panel, Hoecsht 33258 nuclear stain. Third panel, PLC zeta immunostaining. (B) Human sperm immunostained with an antibody raised to a PLC zeta antigenic peptide. First panel, sperm immunostained with PLC zeta antibody. Second panel, Hoecsht 33258 nuclear stain. Third panel, overlay of first and second panels.

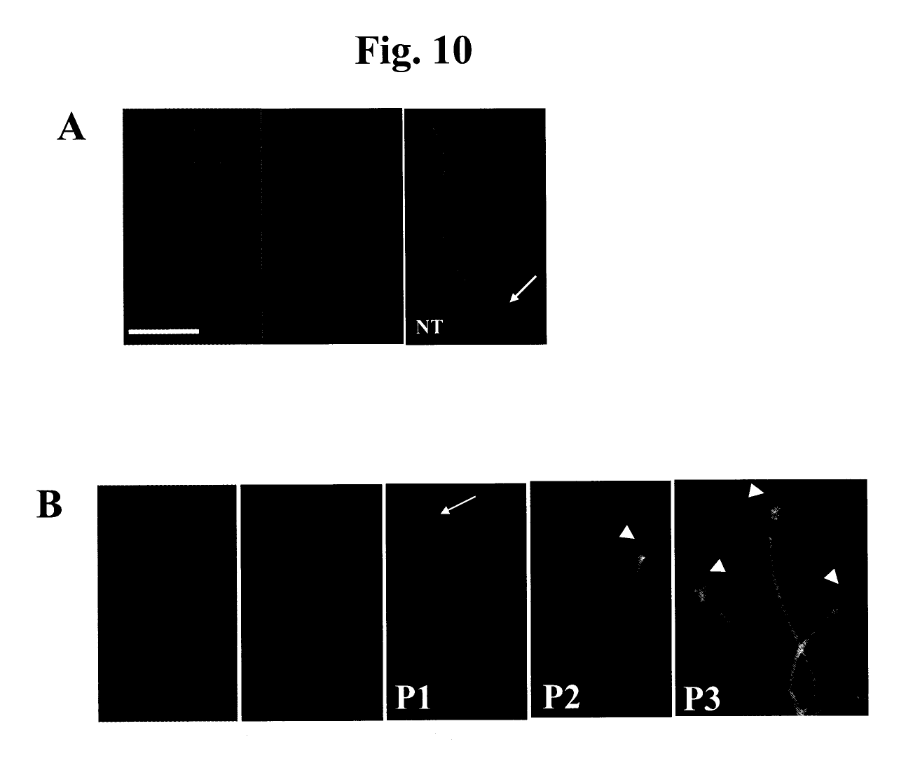

FIG. 10: PLC zeta is present in the equatorial region of control subjects having normal fertility but is absent from this region in patients of low fertility. Immunostaining was used to detect the localization of PLC zeta in human sperm. (A) Control subject having normal fertility exhibits PLC zeta in the equatorial region of the sperm head. First panel, DIC microscopy. Second panel, Hoecsht 33258 nuclear stain. Third panel, PLC zeta immunostain. Arrow indicates equatorial localization of PLC zeta. (B) PLC zeta is absend from the equitorial region of sperm from low fertility patients. First three panels, DIC, Hoecsht 33258, and PLC zeta immunostaining from Patient 1 (P1). Fourth and fifth panels, PLC zeta immunostaining from Patients 2 (P2) and 3 (P3). Arrows indicate equitorial region from which PLC zeta immunostaining is absent. NT indicates the PLC zeta N-terminal antibody.

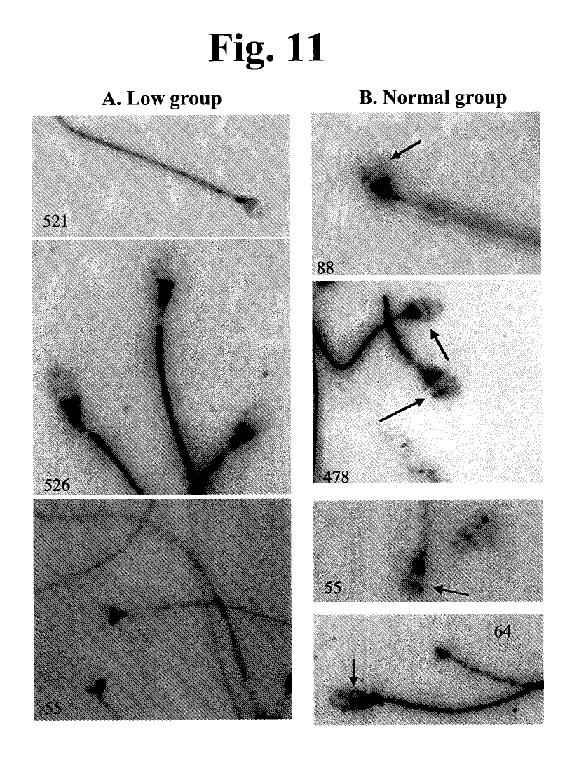

FIG. 11: PLC zeta is present in the equatorial region of control subjects having normal fertility but is absent from this region in patients of low fertility. Immunostaining was used to detect the localization of PLC zeta in human sperm. (A) PLC zeta is absend from the equitorial region of sperm from low fertility patients. The three panels are samples from different patients. (B) Control subject having normal fertility exhibits PLC zeta in the equatorial region of the sperm head (arrows). The three panels are samples from different control subjects.

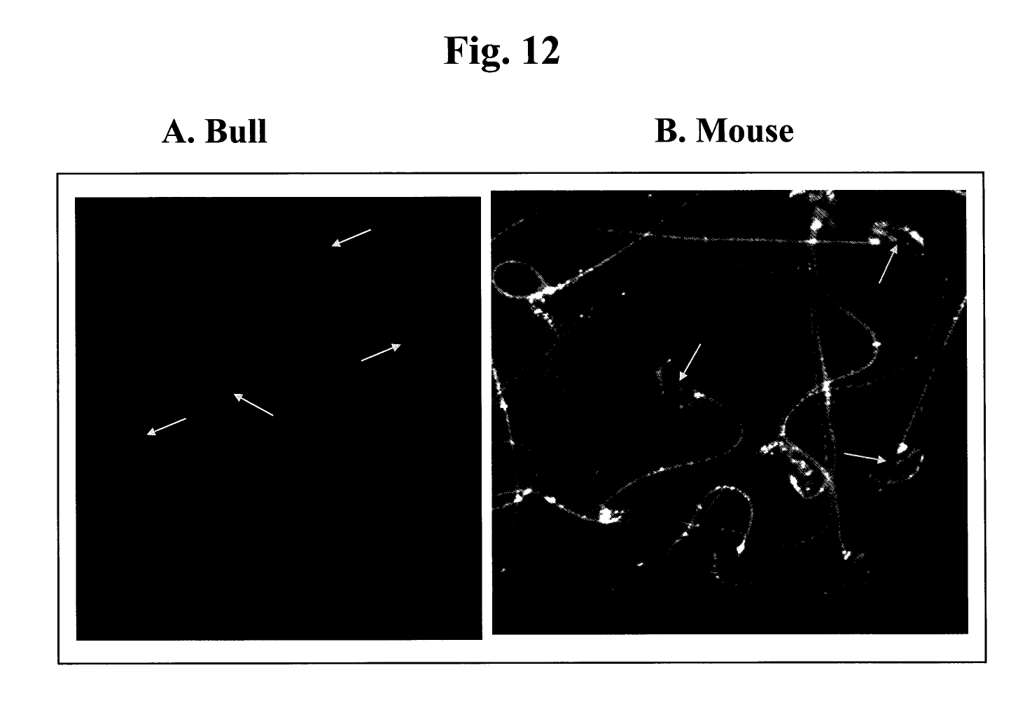

FIG. 12: PLC zeta has species-specific localization in the sperm head. Immunostaining was used to detect PLC zeta localization. (A) Bull sperm exhibit PLC zeta localized to an equatorial band. (B) mouse sperm exhibit PLC zeta localized to a hemispheric section plus a region covering the sperm tip. Arrows indicate PLC zeta localization.

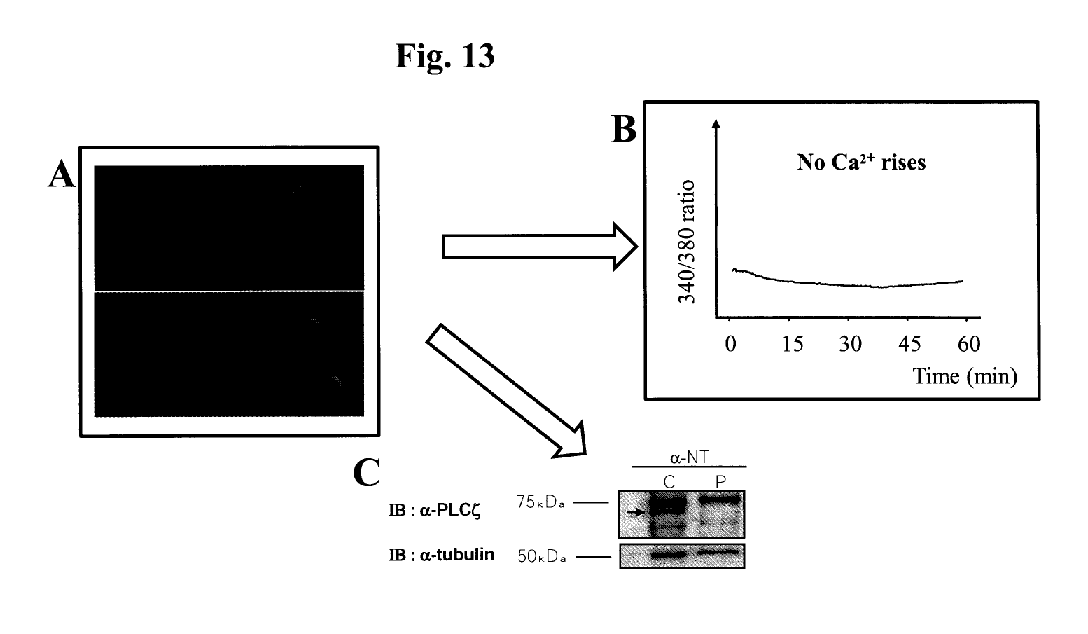

FIG. 13: Absence of PLC zeta in sperm of a patient having male infertility. Sperm samples from a patient that had failed ICSI two times (a total of 38 eggs). (A) The normal equatorial PLC zeta localization was undetectable by immunostaining of the patient's sperm. This patient's sperm looks abnormal in shape, although this patient had sperm that showed normal motility (data not shown). (B) Patient sperm failed to induce [Ca2+]i oscillations when injected into mouse oocytes. (C) PLC zeta protein was undetectable in the patient's sperm by western blotting. C=control subject having normal fertility. P=patient having male infertility. Upper panel: immunoblot done with PLC zeta antibody (NT). Lower panel, loading control stained with antibody to tubulin. Arrow indicates PLC zeta.

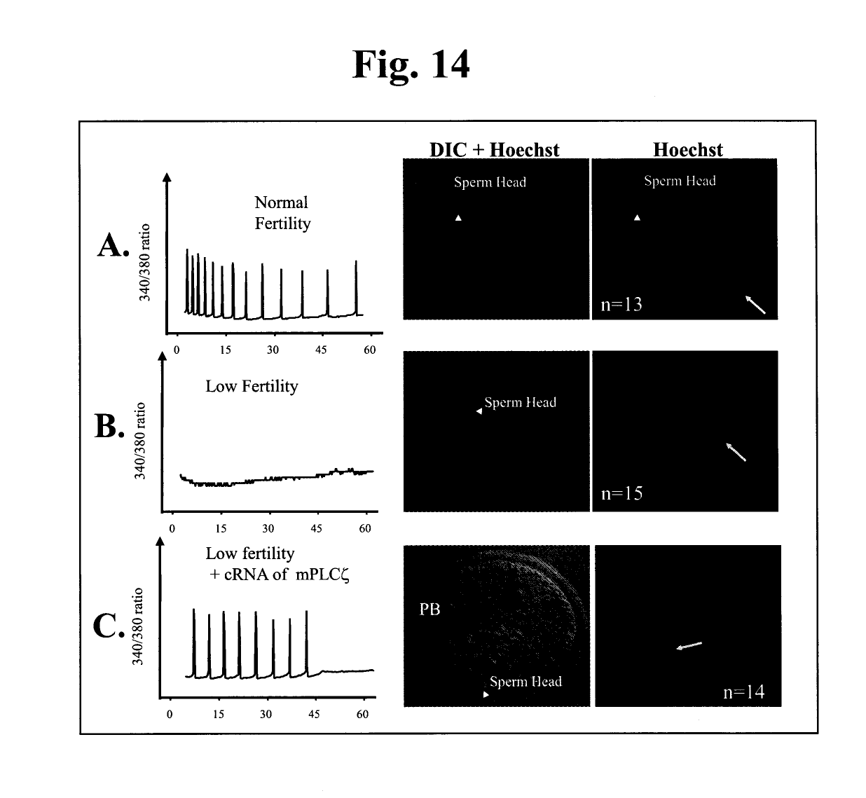

FIG. 14: ICSI failure can be rescued by injection of PLC zeta cRNA. Representative traces of [Ca2+]i oscillations exhibited upon injection of human sperm into mouse eggs are shown for sperm from (A) a control subject known to exhibit normal fertility, (B) a patient with low fertility and having undetectable PLC zeta in sperm, and (C) the same patient when mouse PLC zeta cRNA was also injected into the egg. Differential Interference Contrast (DIC) Microscopy and Hoecsht 33258 fluorescence confirm sperm injection, as shown for representative examples.

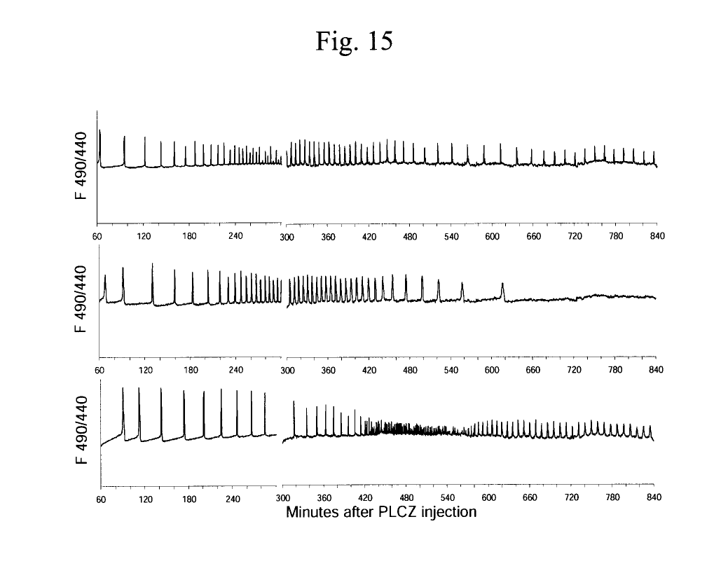

FIG. 15: Representative [Ca²⁺]ᵢ profiles observed in SCNT embryos from 1 to 14 hours after mPLCZ cRNA injection.

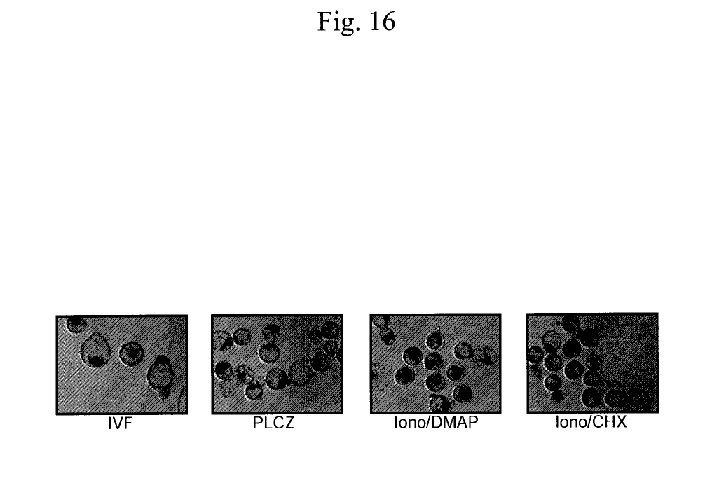

FIG. 16: Representative pictures of blastocysts generated by IVF and SCNT using different activation protocols.

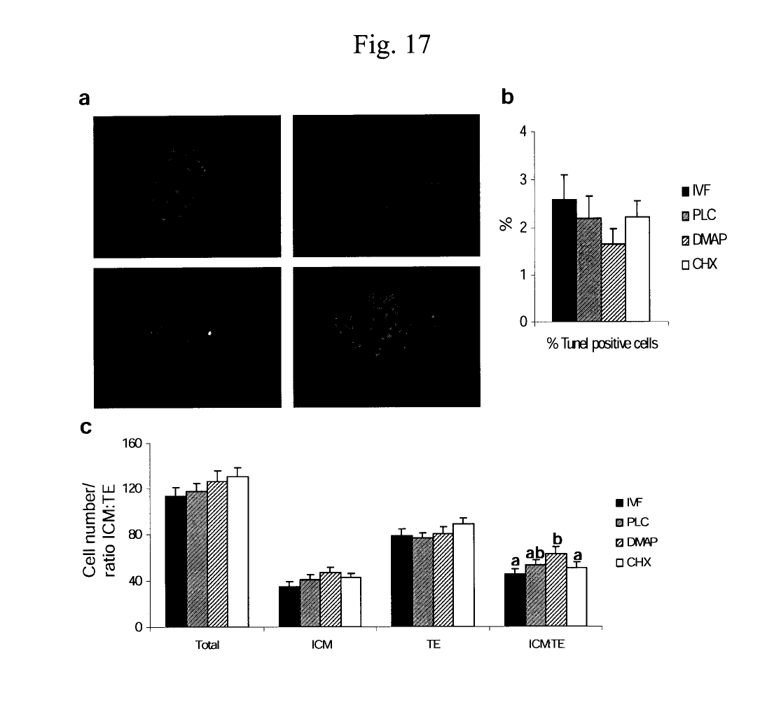

FIG. 17: Cell number, allocation, and apoptosis in IVF and SCNT embryos produced using different activation methods. a) Representative images of analyzed embryos; ICM (blue), TE (red), and TUNEL positive nuclei (Green). b) Quantification of TUNEL positive cells per embryo. c) Comparison of cell number and allocation among groups. ^{a.b}: bars with different superscripts indicate significant differences (P<0.05).

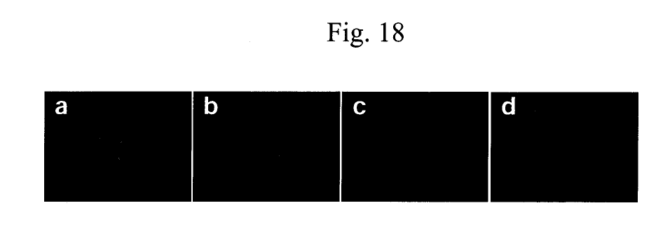

FIG. 18: Representative blastocyst chromosomal spreads. a, b: diploid chromosome complements, c, d: tetraploid chromosome complements.

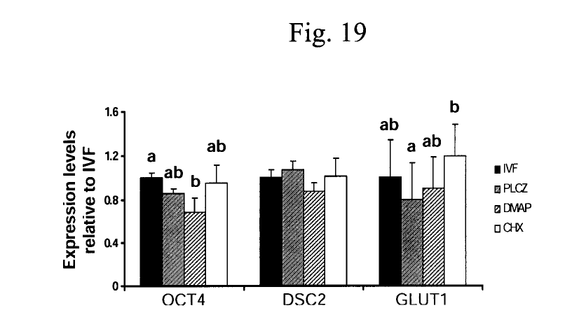

FIG. 19: Quantification of mRNA abundance in 8-cell embryos generated by IVF or SCNT using different activation protocols. ^{a,b}: bars with different superscripts indicate significant differences (P<0.05).

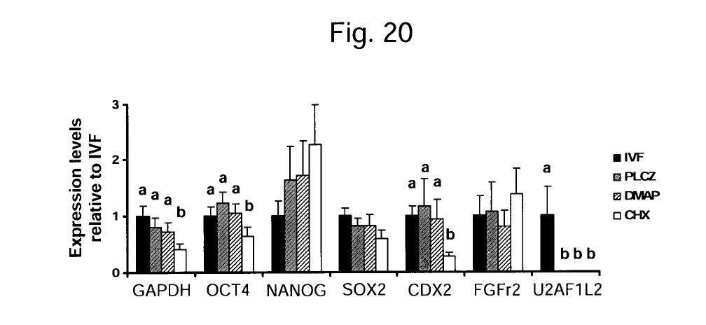

FIG. 20: Quantification of mRNA abundance in blastocysts generated by IVF or SCNT using different activation protocols. a,b: bars with different superscripts indicate significant differences (P<0.05).

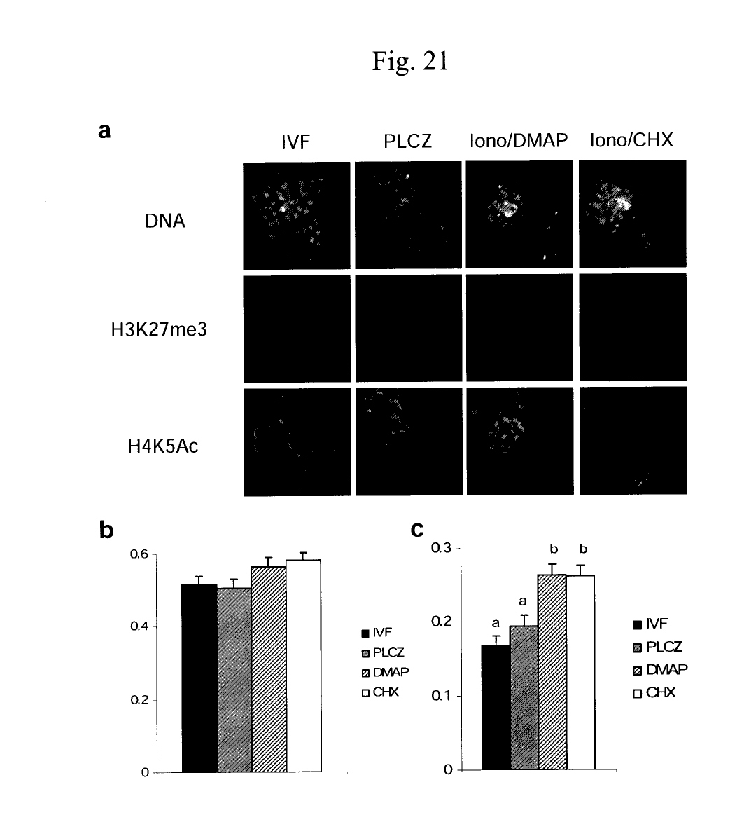

FIG. 21: Immunofluorescense analyses of IVF and SCNT embryos activated by using different protocols. a) Representative pictures of immunostained embryos. Semi-quantitative evaluation of b) H4K5Ac and c) H3K27me3 immunostaining. ^{a,b}: bars with different superscripts indicate significant differences (P<0.05).

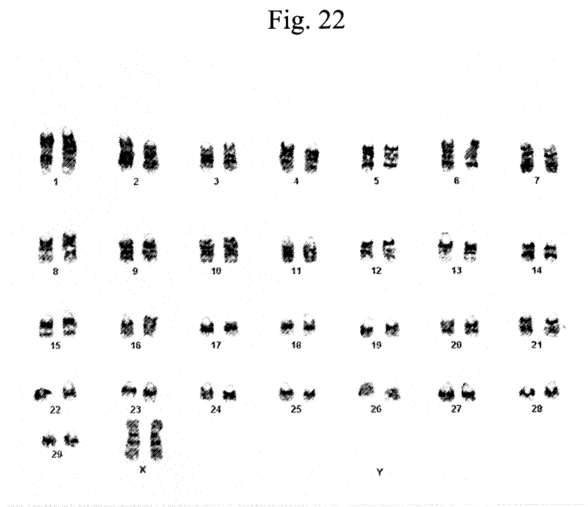

FIG. 22: Cytogenetic analysis of donor cell line used for SCNT. The analysis was performed on G-banded cells by Cell Line Genetics (Madison, Wis.) resulting in an apparently normal Female Bovine Karyotype (60, XX).

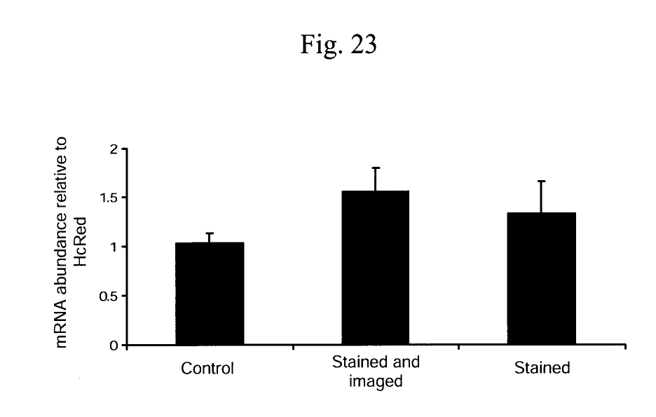

FIG. 23: Confocal imaging of bovine embryos does not affect the level of GAPDH transcript abundance. GAPDH mRNA abundance was analyzed by quantitative real-time RT-PCR in groups of five 8-cell embryos that were stained with Syto 16 (5 μM for 15 minutes, Stained, n=4); stained and imaged using a spinning-disc confocal microscope (Stained and imaged, n=4); or left untreated (Control, n=4). HcRed cRNA was added to the sample before RNA extraction as an external control. No significant differences were observed among the groups (P=0.43).

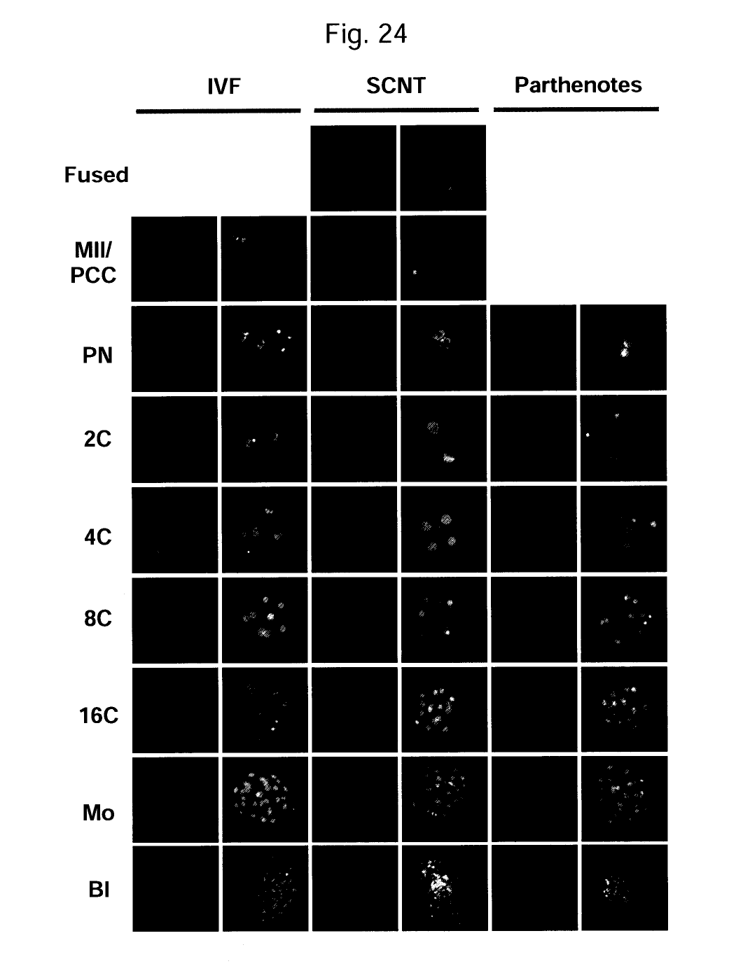

FIG. 24: Immunofluorescence staining for trimethylated Histone 3 lysine 27 (H3K27me3) of bovine embryos of different origins at different stages of preimplantation development. Shown are images of H3K27me3 (red) and nuclear staining with bisbenzimide (white) of representative embryos (400×). IVF: in vitro fertilized embryo; SCNT: somatic cell nuclear transfer; Fused: SCNT reconstructed embryo right after fusion of somatic cell and oocyte; MII: metaphase II stage oocyte (IVF group); PCC: premature chromosome condensation (SCNT group); PN: pronuclear stage embryo; 2C: 2-cell stage embryo; 4C: 4-cell stage embryo; 8C: 8-cell stage embryo; 16C: 16-cell stage embryo; Mo: Morula; BI: Blastocyst.

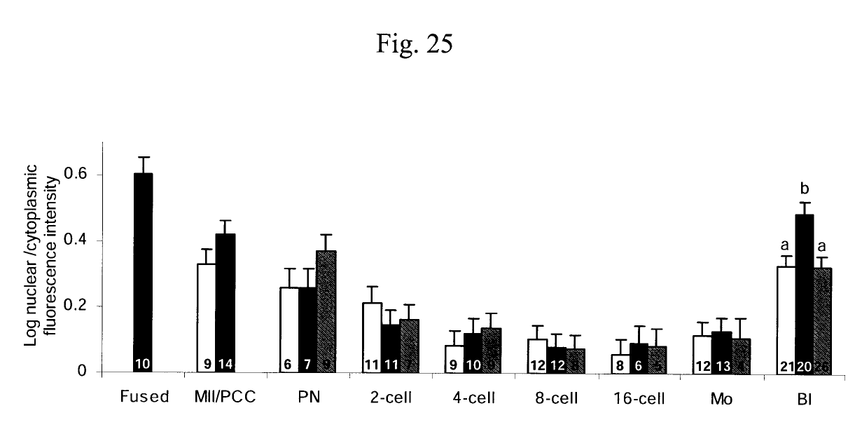

FIG. 25: Level of Histone H3 lysine 27 trimethylation (H3K27me3) in embryos derived from in vitro fertilization (open bars), SCNT (black bars), and parthenogenesis (grey bars) at different stages of preimplantation development. Data shown as Is-mean ±SEM. ^{a,b} Bars with different letters are significantly different (P<0.05). MII=Metaphase II oocyte; PCC=premature chromosome condensation; PN=Pronuclear stage embryo; Mo=Morula; Bl=blastocyst.

### DETAILED DESCRIPTION OF THE INVENTION

The present invention relates to the use of PLC zeta to enhance the efficiency of nuclear transfer and the development of parthenogenic embryos. As described infra, the present inventors have demonstrated for the first time that nuclear transfer embryos (derived from bovine oocytes) may be activated in vitro by the addition of phospholipase C zeta RNA and that the addition thereof promotes the in vitro activation thereof and efficient embryonic development and also that this process is more efficient than previous activation methods such as those using DMAP. This activation may be effected before, simultaneous or after nuclear transfer.

The present invention also relates to use of PLC zeta to detect male infertility. As described herein, the present inventors have demonstrated for the first time that control subjects exhibiting normal fertility have PLC zeta protein localized to an equatorial region of the sperm, whereas some patients exhibiting low fertility show absence of PLC zeta protein in the equatorial region of their sperm, and further have shown reduction or absence of PLC zeta in whole sperm extracts from patients exhibiting male infertility. Aspects of the invention relate to use of PLC zeta to detect male infertility by detection of reduced levels of PLC zeta protein or RNA in patient sperm, detection of altered spatial localization of PLC zeta protein in sperm, or detection of genomic mutations that cause reduction or loss of PLC zeta localization or function. Furthermore, the levels of PLC zeta protein or RNA, or the localization of PLC zeta, may be tested in a sample of a patient's sperm, or in any sperm progenitor, for example Spermatogonia, Spermatocytes, or Spermatids. Furthermore, the levels of PLC zeta protein or RNA, or the localization of PLC zeta may be tested in fertilized oocytes or cells cultured or developed therefrom.

The present invention also relates to detection of male infertility through identification of individuals possessing non-functional allelic variants of phospholipase C zeta. Non-functional allelic variants are any PLC zeta alleles that fail to elicit normal [Ca2+]i fluctuations when introduced into oocytes. These non-functional allelic variants may also be identified as the sequences of phospholipase C zeta observed in males known to have reduced levels of phospholipase C zeta and infertility, or the sequences of PLC zeta observed in males exhibiting infertility that can be successfully overcome through administration of exogenous PLC zeta. Additionally, non-functional allelic variants may be identified as sequences of PLC zeta similar to said non-functional allelic variants of phospholipase C zeta, or if said genomic sequences of a patient's phospholipase C zeta genes encode allelic variants of phospholipase C zeta having mutations that encode truncated phospholipase C zeta protein, or possess mutations within splice donor or splice acceptor sequences, or encode mutations within catalytic residues, or have mutations that would trigger nonsense-mediated decay, or any combination thereof. Non-functional allelic variants of PLC zeta may be identified through sequencing of a patient's entire PLC zeta genomic loci, or through targeted sequencing of portions of a patient's PLC zeta genes, or though genetic testing methods that identify nucleotide polymorphisms associated with non-functional PLC zeta alleles, or any other means of identifying alleles of genes as are known in the art.

The present invention also relates to use of PLC zeta to treat male infertility and/or promote the efficiency of in vitro fertilization. As described herein, the present inventors have demonstrated for the first time that male patients exhibiting low fertility and reduction or absence of PLC zeta protein in sperm fail to elicit the Ca2+ oscillations necessary for egg activation when those sperm are injected to oocytes; however, normal Ca2+ oscillations are restored when PLC zeta cRNA is also injected into those oocytes. Aspects of the invention relate to methods of treating male infertility through the use of PLC zeta. Additional aspects of the invention relate to compositions containing PLC zeta for the treatment of male infertility.

The present invention also relates to methods of identifying compounds that are useful for treatment of infertility, preferably male associated infertility. As shown herein, male infertility can result from a deficiency of PLC zeta and can be treated by administration of PLC zeta to fertilized oocytes. Alternatively, compounds induce the expression of PLC zeta or otherwise stimulate intracellular calcium fluctuations similar to those elicited by PLC zeta may be identified through contacting cells that exhibit fluctuations of intracellular calcium concentration upon introduction of PLC zeta with a candidate compound; and monitoring the intracellular calcium concentration. Compounds that induce fluctuations of intracellular calcium concentration that are similar in frequency and amplitude to the fluctuations of intracellular calcium concentration observed when cells are contacted with phospholipase C zeta could be used as an alternative to PLC zeta for the treatment of male infertility.

Based on these results the invention relates to the use of phospholipase C zeta to induce the activation and development of nuclear transfer cells as well as parthenogenic cells and cells produced during in vitro fertilization procedures. In the context of nuclear transfer cells, activation refers to the process by which the cell initiates embryonic development.

Also, the invention relates to use of PLC zeta to induce activation and development of developmentally impaired embryos which are to be used for production of specific tissues and developmental studies.

Nuclear transfer cells for use in the subject in vitro activation methods may be produced by methods known in the art. In general such methods entail injecting or fusing a somatic cell or the nucleus or chromosomes thereof with an oocyte or embryonic cell which is enucleated before or after nuclear transfer. When the cell that is the source of the nucleus or chromosomes is of a different species than the cell that is the recipient thereof, that nuclear transfer cell is referred to as a cross-species nuclear transfer cell.

Methods for producing nuclear transfer embryos using proliferating or non-proliferating donor cells, e.g., somatic cells and nucleic or chromosomes thereof is generally known and is disclosed in numerous patents. Particularly U.S. Pat. Nos. 6,164,276; 7,071,732; 7,064,248; 6,700,037; 6,600,087; 6,525,243; 6,258,998; 6,252.133; 6,235,969; 6,235,979; 6,215,041; 6,147,276; 6,011,197; 6,436,701; 6,331,659; 6,271,436; 6,258,998; 6,194,202; 6,590,139; 6,635,802; 6,603,453; 6,753,453; 6,808,704; 6,906,238; and 7,053,264 all relate to nuclear transfer cloning methods and materials for use therein. These patents are all incorporated by reference in their entireties herein, as are all references cited herein.

In the present invention the embryos may be derived from any species including human, non-human primate such as cynomolgus monkey, bovine, ovine, equine, canine, feline, caprine, murine, and the like. Typically such embryos are produced by fusing or inserting a proliferating somatic cell such as a fetal fibroblast with an oocyte of the same or different species which may be enucleated before or after nuclear transfer. Preferably the embryos will comprise bovine nuclear transfer embryos. Alternatively such embryos may comprise human embryos produced from donor human oocytes and a somatic cell or nucleus thereof that is fused or inserted therein. The nuclear transfer embryos used in the present invention may be transgenic e.g., by the introduction of a gene encoding a therapeutic human protein.

As noted the embryos used for activation if human for ethical reasons may be genetically mutated, e.g., by removal or duplication of one or more chromosomes or portions of chromosomes and/or by targeted genetic mutations, so that they are incapable of giving rise to a viable fetus. For example the donor nucleus may have mutations that impair neuronal development or which only allow certain tissue lineages types to develop such as endodermal, mesodermal or ectodermal tissue lineages. Thereby the activated embryo will only give rise to specific tissues and cannot be used for human cloning.

In the case of parthenogenic embryos these embryos may be derived from gametes such as primordial germ cells or unfertilized oocytes, or may be derived from fused gametes. As used herein, the term parthenogenetic also encompasses androgenetic cells, such as those derived from sperm or sperm progenitors. For example parthenogenic embryos may be derived from unfertilized bovine, human, non-human primate, murine, bovine or other species oocytes.

In embodiments wherein PLC zeta is used to facilitate in vitro fertilization the embryo will typically comprise a human embryo. This may be helpful in the situation wherein the sperm used for fertilization do not produce sufficient levels of PLC zeta for fertilization and embryonic development to proceed.

In the present invention such parthenogenic embryos or nuclear transfer embryos or in vitro fertilization produced embryos will be activated by providing same with a sufficient amount of phospholipase C zeta. Such phospholipase C zeta may be of any species origin but preferably is of human, non-human primate, rodent or bovine origin. Such PLC zeta enzymes will include the unmodified, i.e., wild type PLC zeta enzyme and functional fragments and variants. In general such variants will comprise at least 80% sequence identity to the sequence of the corresponding native PLC zeta, e.g., human PLC zeta, more typically at least 90 or 95% sequence identity therewith. Functional PLC zeta variants and fragments may be identified in assays which assess whether these variants or fragments promote fertilization or activation, comparably to native PLC zeta.

The present activation methods may be effected by incubating a nuclear transfer or in vitro fertilization produced embryo or other appropriate cell with a sufficient amount of purified phospholipase C zeta enzyme or a functional variant or fragment thereof or by injecting the embryo or oocyte with a nucleic aid sequence (cDNA or RNA) encoding the phospholipase C zeta enzyme or a functional fragment or variant thereof or a vector containing. Additionally, in some instances the somatic cell used to produce the nuclear transfer embryo may be genetically engineered to express the phospholipase C zeta enzyme, e.g., under inducible conditions. Thereby activation may be triggered when the embryo is in a suitable environment. e.g. a uterine environment or a cell culture environment.

As noted above, Parthenogenesis is the development of an embryo without paternal contribution (See Kaufman, M. H., et al., 1975. Genetic control of haploid parthenogenetic development in mammalian embryos. Nature. 254, 694-5). When placed in the uterus of a surrogate mother, mammalian parthenogenetic embryos will develop to different stages depending on the species, but never to term (See Kono, T., 2006. Genomic imprinting is a barrier to parthenogenesis in mammals. Cytogenet Genome Res. 113, 31-5). Bovine oocytes can be parthenogenetically activated using ionomycin, ionophore, ethanol, or electric stimuli (See Alberio, R., et al., 2001. Mammalian oocyte activation: lessons from the sperm and implications for nuclear transfer. Int J Dev Biol. 45, 797-809). All of these compounds will trigger a monotonic [Ca²⁺]ᵢ increase that, while necessary, is not sufficient to completely downregulate the synthesis of Maturation-Promoting Factor (MPF). To accomplish this goal, these [Ca²⁺]ᵢ releasing agents must be used in combination with a protein synthesis or protein kinase inhibitor such as cycloheximide or 6-dimethylaminopurine (DMAP), respectively (See Alberio, R., et al., 2001. Mammalian oocyte activation: lessons from the sperm and implications for nuclear transfer. Int J Dev Biol. 45, 797-809). Using these activation protocols, parthenotes can reach the blastocyst stage at reasonable rates; however, the impact these treatments have on in vivo development has not been studied, mainly because parthenogenetic embryos are inherently limited in their developmental capacity.

In cattle, immunoreactive PLC zeta is found in sperm, and injection of mouse PLC zeta cRNA induces [Ca²⁺]ᵢ oscillation in oocytes (See Malcuit, C., et al., 2005. Fertilization and inositol 1,4,5-trisphosphate (IP3)-induced calcium release in type-1 inositol 1,4,5-trisphosphate receptor down-regulated bovine eggs. Biol Reprod. 73, 2-13). However, the potential of bovine PLC zeta cRNA to induce [Ca²⁺]ᵢ oscillations and parthenogenetic activation of bovine oocytes has not been reported.

We report herein the calcium-oscillation-inducing activity of mouse and bovine PLC zeta cRNA injected into bovine oocytes, as well as their effects on IP3R-1 concentration. We also compared the development of oocytes activated using mouse and bovine PLC zeta cRNA to commonly used chemical activation

For example bovine nuclear transfer embryos may be produced which comprise a phospholipase C zeta gene operably linked to a promoter that is inducible in the presence of a selectable marker. Alternatively cross species embryos may be produced wherein the phospholipase C zeta gene is directly or indirectly linked to an amplifiable marker such as DHFR gene resulting in amplication of the PLC zeta gene under selection conditions. This may enhance the development of such cross species embryos. In particular this is contemplated in the case of cross species embryos derived from human donor cells or nuclei and non-human recipient ooplasts, such as bovine, rabbit or non-human primate ooplasts.

Based on the role of phospholipase C zeta in promoting oocyte activation and embryonic development the invention further relates to the detection of phospholipase C zeta levels in sperm cells and correlating these levels to fertility as a means of detecting male infertility. This may be effected by use of antibodies that specifically bind PLC zeta, which may be labeled with a detectable marker such as a fluorophore or radionuclide or a detectable enzyme or polypeptide that allow for the detection and quantification of PLC zeta levels. Alternatively, PLC zeta mRNA levels may be detected by use of nucleic acid sequences that hybridize to PLC zeta coding sequences. Thereby, couples with infertility problems can determine if the problem is the result of inadequate PLC zeta enzyme.

As used herein, “detecting male infertility” refers to identification of individuals that are infertile, as well as to identification of a cause of infertility in individuals that are already known or suspected to be infertile. “Infertile” refers to any decrease in fertility compared to individuals of normal fertility. As described herein, in the exemplary embodiments male infertility is detected based on the levels of expression of PLC zeta by sperm and/or based on the detection of chromosomal mutations that correlate to PLC zeta associated male infertility.

As well as detecting male infertility the invention provides for methods of treating male infertility. Herein “treating male infertility” refers to individuals wherein it is found that the PLC zeta levels of expression are inadequate for the sperm of these individuals to effect oocyte activation that this may be effectively overcome by adding an exogenous source of PLC zeta before, during or after fertilization of a recipient oocyte with such sperm. This may allow for couples to produce a viable pregnancy without resorting to a different sperm donor.

The exogenous source of PLC zeta may be a PLC zeta protein or a functional fragment or variant thereof or a nucleic acid sequence encoding as noted supra. The species origin of the PLC zeta protein may be the same species or a different species as the oocyte. As mentioned human PLC zeta, non-human primate, rodent and bovine PLC zeta enzymes are preferred sources of such PLC zeta proteins and nucleic acid sequences. The PLC zeta protein may also be an active fragment of PLC zeta or a nucleic acid sequence encoding. An active fragment of PLC zeta is any subsequence of PLC zeta that has PLC zeta activity. “PLC zeta activity” is the ability to elicit [Ca2+]i fluctuations similar to those elicited by the full length PLC zeta when introduced into oocytes. The PLC zeta may be an active variant of a full length PLC zeta or fragment of PLC zeta. An active variant is any form of PLC zeta that may be obtained by mutagenesis of the encoding nucleic acid, including site-directed mutagenesis or random mutagenesis, or by post-translational or chemical modification of the protein, so long as that form retains PLC zeta activity. The exogenous source of PLC zeta may also be a nucleic acid or another compound that induces the oocyte to produce any full length PLC zeta protein or active fragment or variant thereof described in this paragraph. That nucleic acid may be an RNA whose translation results in a PLC zeta protein or an active fragment or variant thereof. That nucleic acid may also be a DNA sequence encoding a PLC zeta protein or an active fragment or variant thereof. That DNA sequence may be under the control of an inducible or constitutive promoter sequence.

A cell may be induced to express PLC zeta or an active fragment or variant thereof through introduction of nucleic acid sequences by means known in the art. Those means include viral vectors, non-viral vectors, and other means.

Suitable viral vectors include lentiviruses, retroviruses, herpes viruses, adenoviruses, adeno-associated viruses, vaccinia virus, baculovirus, and other recombinant viruses with desirable cellular tropism. Methods for constructing and using viral vectors are known in the art (e.g., Miller and Rosman, BioTechniques, 1992, 7:980-990). Preferably, the viral vectors are replication-defective, that is, they are unable to replicate autonomously in the target cell. Preferably, the replication defective virus is a minimal virus, i.e., it retains only the sequences of its genome which are necessary for encapsulating the genome to produce viral particles. DNA viral vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Examples of particular vectors include, but are not limited to, a defective herpes virus 1 (HSV1) vector (Kaplitt et al., Molec. Cell. Neurosci., 1991, 2:320-330), defective herpes virus vector lacking a glyco-protein L gene, or other defective herpes virus vectors (PCT Publication Nos. WO 94/21807 and WO 92/05263); an attenuated adenovirus vector, such as the vector described by Stratford-Perricaudet et al. (J. Clin. Invest., 1992, 90:626-630; see also La Salle et al., Science, 1993, 259:988-990); and a defective adeno-associated virus vector (Samulski et al., J. Virol., 1987, 61:3096-3101; Samulski et al., J. Virol., 1989, 63:3822-3828; Lebkowski et al., Mol. Cell. Biol., 1988, 8:3988-3996), each of which is incorporated by reference herein in its entirety.

Suitable non-viral vectors include lipofection or other transfection facilitating agents (peptides, polymers, etc.). Synthetic cationic lipids can be used to prepare liposomes for in vivo transfection of a gene encoding a marker (Felgner, et. al., Proc. Natl. Acad. Sci. U.S.A., 1987, 84:7413-7417; Felgner and Ringold, Science, 1989, 337:387-388; see Mackey, et al., Proc. Natl. Acad. Sci. U.S.A., 1988, 85:8027-8031; Ulmer et al., Science, 1993, 259:1745-1748). Useful lipid compounds and compositions for transfer of nucleic acids are described in PCT Patent Publication Nos. WO 95/18863 and WO 96/17823, and in U.S. Pat. No. 5,459,127. Lipids may be chemically coupled to other molecules for the purpose of targeting (see Mackey, et. al., supra). Targeted peptides, e.g., hormones or neurotransmitters, and proteins such as antibodies, or non-peptide molecules could be coupled to liposomes chemically. Other molecules are also useful for facilitating transfection of a nucleic acid in vivo, such as a cationic oligopeptide (e.g., PCT Patent Publication No. WO 95/21931), peptides derived from DNA binding proteins (e.g., PCT Patent Publication No. WO 96/25508), or a cationic polymer (e.g., PCT Patent Publication No. WO 95/21931).

The nucleic acid may also be introduced by other means known in the art, e.g., electroporation, microinjection, cell fusion, DEAE dextran, calcium phosphate precipitation, use of a gene gun, or use of a DNA vector transporter (e.g., Wu et al., J. Biol. Chem., 1992, 267:963-967; Wu and Wu, J. Biol. Chem., 1988, 263:14621-14624; Canadian Patent Application No. 2,012,311; Williams et al., Proc. Natl. Acad. Sci. USA, 1991, 88:2726-2730). Receptor-mediated nucleic acid delivery approaches can also be used (Curiel et al., Hum. Gene Ther., 1992, 3:147-154; Wu and Wu, J. Biol. Chem., 1987, 262:4429-4432). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al., C. P. Acad. Sci., 1988, 321:893; PCT Publication Nos. WO 99/01157; WO 99/01158; WO 99/01175).

In order to describe the invention the following examples are provided:

### EXPERIMENTAL EXAMPLES

### Materials and Methods

The following materials and methods were used in the experiments described below.

### Chemicals Used in Experiments

All chemicals were purchased from Sigma (St. Louis, Mo.) unless otherwise specified.

### Oocyte Collection and Maturation

Bovine ovaries were obtained from a slaughterhouse and transported in physiological saline solution in an insulated container. Upon arrival at the laboratory, the ovaries were rinsed first with warm tap water and then with physiological saline solution. Antral follicles (2 to 8 mm in diameter) were aspirated using an 18-gauge needle into a 50 mL conical tube by applying 60 mm Hg of negative pressure using a vacuum pump (Cook, Australia). Cumulus oocyte complexes (COCs), with evenly granulated oocyte cytoplasm surrounded by more than four compact layers of cumulus cells, were selected and washed three times in HEPES-buffered HECM medium (See Seshagiri, P., Bavister, B., 1989. Phosphate is required for inhibition by glucose of development of hamster 8-cell embryos in vitro. Biol Reprod. 40, 607-614) (HH; 114 mM NaCl, 3.2 mM KCl, 2 mM CaCl2, 0.5 mM MgCl2, 0.1 mM Na pyruvate, 2 mM NaHCO3, 10 mM HEPES, 17 mM Na lactate, 1× MEM nonessential amino acids, 100 IU/mL penicillin G, 100 μg/mL streptomycin, 3 mg/mL BSA). COCs were then matured in Medium 199 supplemented with 10 percent FBS (HyClone, Logan, Utah), 1 μg/mL of FSH (Sioux Biochem, Sioux City, Iowa), 1 μg/mL of LH (Sioux Biochem, Sioux City, Iowa), 1 μg/mL 17β-estradiol, 2.3 mM of sodium pyruvate, and 25 μg/mL of gentamicin sulphate (Gibco, Grand Island, N.Y.).

PLC Zeta Complementary RNA (cRNA) Preparation

A pBluescript vector containing the full-length coding sequence of murine PLC zeta or pGEMT-easy vector of bovine PLC zeta was linearized with EcoR1 for mouse PLC zeta and Sal I for bovine PLC zeta and used as template for in vitro transcription by the T7 mMessage mMachine High Yield Capped RNA Transcription Kit (Ambion, Austin, Tex.), following manufacturer instructions. Then, a poly-A tail was added to the cRNA using the Poly(A) Tailing Kit (Ambion). Finally, the cRNA was purified using the Mega Clear Kit (Ambion) and stored at −80° C. in single-use aliquots.

Just before use, the cRNA was thawed on ice, heated to 85° C. for three minutes, and centrifuged at 13,000 RPM at 4° C. for five minutes. Then appropriate dilutions in RNAse-/DNAse-free water (Ambion) were prepared.

cRNA Microinjection

For cRNA injection in FIGS. 1 through 5, 15 through 25, and bovine oocytes in FIGS. 6 through 14, a Petri dish containing a 1 μL drop of cRNA and a 50 μL drop of HH under mineral oil was placed on an inverted microscope (TE2000-U, Nikon, Japan) equipped with micromanipulation equipment (Narishige, Japan) at room temperature. The oocytes were placed in the HH media and injected using a beveled micropipette (5 μm internal diameter, MIC-50-0, Humagen, Charlottesville, Va.) loaded with Fluorinert, using hydraulic microinjection equipment (Eppendorf, Westbury, N.Y.). cRNA was loaded from the tip of the pipette each time before microinjection. Then, the pipette was advanced into the oocytes, and the cytoplasm was aspirated by applying negative pressure to ensure plasma membrane breakage. Finally, the aspirated cytoplasm, followed by the cRNA, was injected into the oocyte by applying positive pressure. The amount of PLC zeta cRNA injected was controlled by observing the meniscus at the cRNA-Fluorinert interface. Injection volume was approximately 6 picoliters except where indicated otherwise.

For cRNA injection into mouse oocytes in FIGS. 6 through 14, a picoinjector system was used. Eggs were microinjected as previously described (Kurokawa et al., 2004. Evidence that activation of Src family kinase is not required for fertilization-associated [Ca2+]i oscillations in mouse eggs. Reproduction 127, 441-454). Reagents were diluted in injection buffer [75 mM KCl and 10 mM HEPES (pH 7.0)], loaded into glass micropipettes and delivered by pneumatic pressure (PLI-100 picoinjector, Harvard Apparatus, Cambridge, Mass.). Each egg received 7-12 pl (1-3% of the total volume of the egg). pBluescript containing the full-length coding sequence of mouse PLC (a gift from Dr K. Fukami, Tokyo University of Pharmacy and Life Science, Japan) downstream of a T7 promoter was in vitro transcribed using the T7 mMESSAGE mMACHINE Kit (Ambion, Austin, Tex.), as reported previously (See Kurokawa et al., 2004. Evidence that activation of Src family kinase is not required for fertilization-associated [Ca2+]i oscillations in mouse eggs. Reproduction 127, 441-454).

Injection of Sperm into Oocytes

Sperm was centrifuged, and 100 microliters of microinjection buffer (MIB) (75 mM KCl and 20 mM HEPES, pH 7.0) was added and frozen at minus 80 degrees Celsius until use. Sperm was washed three times with MIB after thawing. One part sperm suspension was mixed with one part MIB additionally containing 12% polyvinylpyrrolidone (PVP, Mr=360 kDa: Sigma). ICSI was carried out as previously described (Kimura and Yanagimachi, 1995; See Kurokawa and Fissore, 2003. ICSI-generated mouse zygotes exhibit altered calcium oscillations, inositol 1,4,5-trisphosphate receptor-1 down-regulation, and embryo development. Mol Hum Reprod. September; 9(9):523-33) using Narishige manipulators (Medical System Co., USA) mounted on a Nikon diaphot microscope (Nikon Inc., USA). ICSI was performed in hCZB medium at room temperature. Whole sperm was delivered into the egg's cytosol using a piezo micropipette-driven unit (Piezodrill: Burlegh Instruments Inc, USA).

### Intracellular Calcium Monitoring

In FIGS. 1 through 5, matured oocytes were injected with 0.5 mM Fura-2 dextran (MW 10,000, Molecular Probes, Eugene, Oreg.) as described for PLC zeta cRNA. Oocytes were monitored in groups in 100 μL drops of protein-free TL-Hepes medium placed on a Petri dish with a glass bottom and covered with mineral oil. A 75 W Xenon arc lamp provided the excitation light and excitation wavelengths were of 340 and 380 nm. Wavelengths greater than 510 nm were collected through a 20× objective by Photometrics CCD SensSys camera (Roper Scientific; Tucson, Ariz.). Fluorescent intensity ratios (340/380 nm) were measured every twenty seconds for up to three hours using the software SimplePCI (C-Imaging System, Cramberry Township, Pa.).

In FIGS. 6 through 14, [Ca2+]i measurements were carried out as previously described (Kurokawa and Fissore, 2003). Briefly, eggs were loaded with 1 μM Fura-2 acetoxymethylester (Fura-2 AM; Molecular Probes, Eugene, Oreg., USA) supplemented with 0.02% pluronic acid (Molecular Probes) for 20 min at room temperature. Oocytes were monitored simultaneously using a 20× objective on a Nikon Diaphot inverted microscope (Nikon Corp., Tokyo, Japan) fitted for fluorescence measurements. Excitation light was provided by a 75 W Xenon lamp. The excitation wavelength was alternated between 340 and 380 nm by a filter wheel (Ludl Electronic Products, Hawthorne, N.Y., USA), and emitted light was passed through a 510 nm barrier filter and collected with either a cooled Photometrics SenSys CCD camera or a cool SNAP ES digital camera (Roper Scientific, Tucson, Ariz., USA). SimplePCI software (Compix Imaging Inc., Cranberry, Pa., USA) was used to monitor [Ca2+]i and synchronize filter wheel rotation. [Ca2+]i values were reported as the ratio of 340/380 nm fluorescence. Fluorescence ratios were obtained every 10 or 20 s. All [Ca2+]i measurements were conducted on a warming stage (36° C.) using TL-HEPES medium.

In FIGS. 15 through 25, intracellular Ca²⁺ concentration was measured using Fura red. After PLCZ injection, the zygotes were loaded in HH medium containing 2 μM Fura red-AM (Invitrogen), 0.02% Pluronic F-127 (Invitrogen) and 0.5 M sulfinpyrazone for 10 min at 38.5° C. After loading, oocytes were placed in 50 μL drops of protein-free HH medium containing 0.5 M sulfinpyrazone on a Petri dish with a glass bottom and covered with mineral oil. During the first 5 hours after PLCZ injection the medium was also supplemented with 7.5 μg/mL of cytochalasin B. The Petri dish was placed on a heated stage on a Nikon TE2000-U microscope (Nikon, Tokio, Japan). A 120 W metal halide lamp (X-Cite 120) provided the excitation light through fiber optics and excitation wavelengths were of 440 and 490 nm. Wavelengths greater than 600 nm were collected through a 20× objective by an EMCCD camera fitted with on-chip multiplication gain (Cascade 512B, Roper Scientific). Fluorescent intensity ratios (440/490 nm) were measured every twenty seconds using Metamorph software (Universal Imaging Corp., Downingtown, Pa.).

### Western Blot of PLC Zeta

The sperm was washed twice with PBS, counted for sperm concentration, and an aliquot containing 500,000 sperm was added to a tube and mixed with an equal volume of 2× sample buffer (Laemmli, 1970) and stored at minus 80 degrees Celsius until use. Samples were boiled for 3 min and loaded into 10% SDS-polyacrylamide gels. The separated proteins were transferred onto PVDF membrane using a Mini Trans Blot Cell (Bio-Rad, Hercules, Calif.) for 1 hr at 4 degrees Celsius. The membranes were first washed in PBS with 0.05% Tween (PBS-T) and then blocked in 6% nonfat dry milk in PBS-T for 1 h. After several washes in PBS-T, the membranes were incubated with a primary antibody (anti-NT, 1:1000) for overnight at 4 degrees Celsius. After washing three times with PBS-T, the membrane was incubated with a horseradish-peroxidase-labeled secondary antibody (Bio-Rad). Immunoreactivity was detected using chemiluminescence reagent (NEN Life Science Products, Mass.) and the Kodak Image Staiton 440CF (NEN).

### Western Blot of IP3R-1

To assess the down-regulation of IP3R-1 protein, cell lysates from 5 bovine eggs were mixed with 15 μl of 2×SB (Laemmli 1970), as described previously (Jellerette et al. 2004), and stored at −80° C. Thawed samples were boiled for 3 min and loaded onto NuPAGE Novex 3-8% Tris-Acetate gels (Invitrogen, Carlsbad, Calif.). After electrophoresis, proteins were transferred onto nitrocellulose membranes (Micron Separations, Westboro, Mass.). The membranes were blocked by incubation in PBS containing 0.1% Tween (PBST) supplemented with 5% non-fat dry milk for 1.5 h at room temperature and then incubated overnight with a rabbit polyclonal antibody raised against the C-terminal amino acids 2735-2749 of mouse IP₃R-1 (Rbt03) (See Parys, J. B., et al., 1995. Rat basophilic leukemia cells as model system for inositol 1,4,5-trisphosphate receptor IV, a receptor of the type II family: functional comparison and immunological detection. Cell Calcium. 17, 239-49). The membranes were subsequently washed in PBST and incubated for 1 h with a goat anti-rabbit secondary antibody conjugated with horseradish peroxidase. Membranes were incubated for 1 minute in cheminoluminescence reagent (NEN Life Science Products, Boston, Mass.) and developed according to manufacturer's instructions. The intensities of IP₃R-1 bands were assessed using a Kodak 440 Image Station (Rochester, N.Y.) and plotted using Sigma Plot (Jandel Scientific Software, San Rafael, Calif.). The intensity of the IP₃R-1 band from bovine MII eggs was given the value of 1 and values in other lanes were expressed relative to this band.

### In Vitro Fertilization

COCs matured for 24 hours were co-incubated with sperm (10⁶ spermatozoa/mL) in a fertilization medium consisting of IVF-TALP (Tyrode's solution) (See Parrish, J. J., et al., 1986. Bovine in vitro fertilization with frozen-thawed semen. Theriogenology. 25, 591-600) supplemented with 10 mM sodium lactate, 1 mM sodium pyruvate, 6 mg/ml BSA, 50 μg/mL heparin, 40 μM hypotaurine, 80 μM penicillamine, and 10 μM epinephrine) at 38.5° C. in 5% CO₂ in air for 20 hours. Presumptive zygotes were vortexed for two minutes to separate cumulus cells. Groups of 40 to 50 presumptive zygotes were cultured in 400 μL drops of KSOM (Chemicon, Temecula, Calif.) supplemented with 3 mg/mL BSA under mineral oil at 38.5° C., 5% CO₂ in air, and humidity to saturation. Seventy-two hours after insemination, 5% FBS was added to the culture media.

### Chemical Parthenogenetic Activation

Oocytes that had matured for 20 to 22 hours were separated from the surrounding cumulus cells by vortexing in HH medium containing hyaluronidase (1 mg/mL) for 5 minutes. MII oocytes were selected based on the presence of a polar body. Twenty-four hours postmaturation, oocytes were exposed to 5 μM ionomycin (Calbiochem, San Diego, Calif.) in HH medium for four minutes, then rinsed three times in HH medium and allocated to either four hours culture in 2 mM DMAP in KSOM or six hours culture in 10 μg/mL cycloheximide (CHX) and 5 μg/mL cytochalasin B in KSOM. After these treatments, oocytes were rinsed five times in HH media and cultured as described for IVF embryos.

### Chromosomal Analysis

In FIGS. 1 through 5, seventy-two hours after activation/fertilization, eight- to sixteen-cell embryos were cultured in KSOM-BSA plus 5% FBS containing colcemid for 12 to 14 hours. Then, embryos were exposed to a hypotonic 1% sodium citrate solution for three to five minutes to induce nuclear swelling. Subsequently, embryos were placed on a clean glass slide in a small volume of media. A methanol-acetic acid solution (1:1) was dropped on the embryos while gently blowing with the slides placed under the stereoscope. Then, just before the solution dried, another drop of methanol-acetic acid solution was placed on the embryos and allowed to dry for at least 24 hours at room temperature. After drying, slides were stained with 5% Giemsa solution (Invitrogen, Carlsbad, Calif.) for ten minutes. Chromosome spreads were evaluated at X1000 magnification with oil immersion optics (Nikon, Japan). Embryos were classified as being haploid, diploid, triploid, tetraploid, polyploid, and mixoploid.

In FIGS. 15 through 25, embryos were incubated for 12 to 14 hours in KSOM-BSA plus 5% FBS containing 0.05 μg/mL demecolcine. Then, embryos were exposed to a hypotonic 0.075M KCL solution for five minutes to induce nuclear swelling. Subsequently, embryos were placed on a clean glass slide in a small volume of media. A methanol-acetic acid solution (1:1) was dropped on top of embryos while gently blowing with the slides placed under the stereoscope. Just before the solution dried the slide was submerged in a 3:1 methanol-acetic acid solution for 1 hour, and then allowed to dry at room temperature for 24 hours. After drying, samples were mounted using Prolong Gold antifade solution with diamidino-2-phenylindole (DAPI; Invitrogen). Chromosome spreads were evaluated under epifluorescence at 1000× magnification with oil immersion optics (Nikon, Japan). Embryos were classified as being haploid, diploid, triploid, tetraploid, polyploid, and mixoploid.

### Statistical Analysis

In FIGS. 1 through 5, cleavage and blastocyst rates were analyzed by chi square test when 4 or fewer replicates were available. When more than 4 replicates were available, cleavage and blastocyst rate were analyzed by ANOVA using the general linear model procedure of SAS (Carry, N.C.). Continuous variables were analyzed by ANOVA using the general linear model procedure of SAS and comparisons among treatments performed using contrast statements. The proportion of embryos with abnormal ploidy was analyzed by chi square test.

In FIGS. 15 through 25, quantitative response variables were analyzed by ANOVA using the MIXED procedure of SAS (Carry, N.C.). Rates of embryonic development to cleavage and blastocyst stage were evaluated using the GLIMMIX procedure of SAS.

### Somatic Cell Nuclear Transfer

Oocytes were obtained from slaughterhouse-derived ovaries and matured in vitro as previously described (See, Ross P J, Perez G I, Ko T, Yoo M S, Cibelli J B. Full developmental potential of mammalian preimplantation embryos is maintained after imaging using a spinning-disk confocal microscope. Biotechniques 2006; 41: 741-750). SCNT was performed as described (See, Ross P J, Perez G I, Ko T, Yoo M S, Cibelli J B. Full developmental potential of mammalian preimplantation embryos is maintained after imaging using a spinning-disk confocal microscope. Biotechniques 2006; 41: 741-750). Briefly, 16-18 hours after oocyte maturation the cumulus cells were removed by vortex agitation in media containing 1 mg/mL hyaluronidase. Oocyte enucleation was performed by aspirating the metaphase II chromosomes in a small volume of surrounding cytoplasm. Donor cells were dissociated by treatment with 10 IU/ml of pronase in HECM-Hepes (HH) media (See, Seshagiri P, Bavister B. Phosphate is required for inhibition by glucose of development of hamster 8-cell embryos in vitro. Biol Reprod 1989; 40: 607-614) for 5 minutes. A single cell was inserted into the perivitelline space of the enucleated oocyte and fused in calcium-free sorbitol fusion medium by applying a single direct current pulse of 234 volts/mm for 22 μs.

### Activation and Embryo Culture

Activation of fused NT units was performed 2 hours after fusion. Three different activation protocols were implemented: 1) lonomycin/DMAP, 2) lonomycin/CHX and 3) PLCZ. In groups 1 and 2, the embryos were treated with 5 μM ionomycin (Calbiochem, San Diego, Calif.) for 4 minutes followed by incubation in KSOM medium containing either 10 μg/mL cycloheximide and 5 μg/mL cytochalasin B for 5 hours (lonomycin/CHX), or 2 mM 6-DMAP from 4 hours (lonomycin/DMAP). Activation using PLCZ was performed by intracytoplasmic injection of ˜6-8 μL of 1 μg/μL mPLCZ cRNA as previously described (Chapter 3). Then the embryos were cultured in potassium simplex optimized medium (KSOM) containing 7.5 μg/μL cytochalasin B for 5 hours to prevent the extrusion of the second polar body. After activation, the NT units were rinsed several times in hepes buffered-HECM (HH) medium and cultured in 400 μL drops of KSOM medium supplemented with 3 mg/mL of bovine serum albumin (BSA) under mineral oil at 38.5° C. and 5% CO2 in air. On day 3 (NT=day 0), the embryo culture drops were supplemented with 10% fetal bovine serum (FBS) and cultured under the same conditions until day 7. Eight-cell and blastocyst stage embryos were collected 50 and 180 h after activation respectively.

Fertilized control embryos were produce by in vitro fertilization using tyrodes albumin lactate pyruvate (TALP)-based medium (See, Parrish J J, Susko-Parrish J L, Leibfried-Rutledge M L, Critser E S, Eyestone W H, First N L. Bovine in vitro fertilization with frozen-thawed semen. Theriogenology 1986; 25: 591-600).

### Blastocysts Differential Staining and TUNEL Assay

The zona pellucida of each blastocyst was removed by incubation in 10 IU/mL pronase for 2 min. After thoroughly rinsing the embryos in HH medium, they were exposed for 10 seconds to 0.2% Triton X-100 in PBS containing 2 mg/mL BSA. The embryos were then incubated 15 minutes in PBS-BSA containing 10 μg/mL bisbenzimide and 30 μg/mL propidium iodide. After staining, the embryos were fixed in 4% paraformaldehyde for 15 minutes and stored at 4° C. for no more than 7 days until terminal deoxynucleotidyl transferase-mediated dUTP nick-end labeling (TUNEL) assay was performed. TUNEL labeling was performed using the In Situ Cell Death Detection Kit (Roche Applied Science) following manufacturer indications. Briefly, the embryos were exposed to the labeling solution, containing the terminal deoxynucleotidyl transferase and fluorescein-labeled nucleotide mixture, for 1 hour at 37° C. in a humid chamber. The embryos were then treated with RNase A (50 IU/mL) for 30 min at 37° C. RQ1-DNase (10 IU/mL)-treated embryos were used as a positive control and negative controls were incubated in labeling solution omitting the enzyme. After intensive washing in PBS-BSA, the embryos were mounted in a small drop of ProLong Gold antifade solution (Invitrogen) and evaluated under epifluorescence microscopy. Trophectoderm cells were observed as red nuclei, inner cell mass cells as blue nuclei, and TUNEL positive cells as green nuclei.

### Cell Number Determination of Live Embryos

The total number of cells in the blastocysts used for gene expression analysis was determined by live confocal microscopy as previously described (See, Ross P J, Perez G I, Ko T, Yoo M S, Cibelli J B. Full developmental potential of mammalian preimplantation embryos is maintained after imaging using a spinning-disk confocal microscope. Biotechniques 2006; 41: 741-750). Briefly, the nuclei were stained by incubation in HH medium containing 5 μM Syto 16 (Molecular Probes, Eugene, Oreg.) for 15 min. Then, the embryos were placed with the ICM facing the objective lens in between two coverslips separated from each other by 150 μm and imaged using a spinning-disk confocal system (CARV, Atto Bioscience Inc. Rockville, Md.) mounted on a Nikon TE2000-U microscope. A Z-stack of the embryo was acquired every 5 μm and the images were processed for 3-D deconvolution using Autoquant and analyzed using Metamorph software. All nuclei were marked by drawing a contour on the image for each focal plane and counted.

### Quantitative RT-PCR

Groups of 5 8-cell embryos and individual blastocysts were lysed in 20 μL of extraction buffer, and then incubated at 42° C. for 30 min followed by centrifugation at 3000 g for 2 minutes and stored at −80° C. Before RNA extraction, each sample was spiked with 2 μL of 250 fg/μl of HcRed1 cRNA, used as an exogenous control (See, Bettegowda A, Patel O V, Ireland J J, Smith G W. Quantitative analysis of messenger RNA abundance for ribosomal protein L-15, cyclophilin-A, phosphoglycerokinase, beta-glucuronidase, glyceraldehyde 3-phosphate dehydrogenase, beta-actin, and histone H2A during bovine oocyte maturation and early embryogenesis in vitro. Mol Reprod Dev 2006; 73: 267-278), and 50 μg of tRNA as a carrier. Total RNA was extracted form each sample using the PicoPure RNA Isolation Kit (Arcturus) according to the manufacturer's instructions. Residual genomic DNA was removed by DNAse I digestion using an RNAse-Free DNAse Set (Quiagen). RNA was eluted from the purification column using 11 μL of nuclease-free water (Ambion). RNA was then primed with oligo-dT (Invitrogen) and converted into cDNA using Superscript II (Invitrogen) following manufacturer's instructions. Each reverse transcription reaction (RT) was finally diluted with nuclease free water to a final volume of 60 μL.

The quantification of all gene transcripts was done by real-time quantitative RT-PCR using SYBR Green PCR Master Mix (Applied Biosystems, Foster City, Calif.). Absolute quantification using this method is described elsewhere (Li and Wang, 2000; Whelan et al., 2003). Primer sequences for all the genes are shown in Table 5.

Each reaction mixture consisted of 2 μL of cDNA, 5 μmol of each forward and reverse primers, 7.5 μL of nuclease free water, and 12.5 μL of SYBR Green PCR Master Mix in a total reaction volume of 25 μL. Reactions were performed in duplicate for each sample in an ABI Prism 7000 Sequence Detection System (Applied Biosystems). Dissociation curves were performed after each PCR run to ensure that a single PCR product had been amplified.

The copy number of HcRed1 cRNA was determined for each sample using a standard curve constructed from the plasmid pHc-Red1-Nuc. For HcRed, GAPDH, OCT-4, NANOG, SOX2, CDX2 and FGFr2 plasmids containing the partial cDNAs were used to construct standard curves using tenfold serial dilutions. For TRYP8, GLUT1, DSC2 and U2AF1L2 a relative standard curve was used to determine abundance in arbitrary units using serial dilutions of amplified cDNA from a pool of bovine IVF and SCNT blastocysts and fibroblasts.

For each measurement, threshold lines were adjusted to intersect amplification lines in exponential portion of amplification curve using the automatic setting of the thermocycler program. HcRed1 (external control) abundance was determined in each sample and used to normalize for differences in RNA extraction and RT efficiency. Blastocyst embryo samples were further normalized to the total cell number of each individual embryo.

### Immunostaining of Embryos

Embryos were washed in PBS containing 1 mg/mL of PVA, fixed with 4% paraformaldehyde for 15 min in PBS (GIBCO) and stored at 4° C. in PBS containing 1 mg/mL of polyvinyl alcohol (PVA) for no longer than 3 weeks. Embryos were permeabilized in 1% Triton X-100 for 30 min at room temperature, then incubated with Image-iT FX signal enhancer (Invitrogen) for 30 min, and blocked with 10% normal goat serum for 2 hours. Embryos were incubated overnight at 4° C. in 1% BSA and primary antibodies against trimethylated lysine 27 of histone H3 (H3K27me3; Abcam, ab6002) and acetylated lysine 5 of histone H4 (H4K5Ac; Upstate, 07-327). After 6 h washing in PBS containing 0.1% Triton X-100, embryos were incubated with secondary antibodies conjugated with Alexa 488 and Alexa 594 (Invitrogen) during 1 hour at room temperature. DNA was visualized by bisbenzimide staining. For imaging, embryos were mounted in 11 μl of anti-fading solution and compressed with a coverslip. Imaging was performed using a spinning disk confocal system mounted on a Nikon TE-2000 microscope at 40× (numerical aperture (NA) 1.3) and 100× (NA 1.3) magnifications. Optical sections every 1 μm were acquired for each embryo. Metamorph software was used for image acquisition and analysis. All sections were combined by a maximum projection and each nucleus delineated under the blue channel (nuclear staining). Also, two different cytoplasmic areas were delineated to use as background fluorescence. The regions were then transferred to the red and green channels and the average pixel intensity calculated by the software for each region. For analysis, each region's fluorescence intensity was divided by the average of the two cytoplasmic regions.

### Immunostaining of Sperm

Sperm were washed twice with Dulbeco's phosphate buffered saline (dPBS), and the pellets were fixed with PBS containing 3.7% paraformaldehyde (in dPBS, pH 7.4), at 4° C. for 20 min and washed three times with dPBS. The fixed sperm were kept at 4 degrees Celsius until use. The sperm was permeabilized with 0.1% (v/v) Triton X-100 in dPBS for 5 min, at room temperature. Sperm were then washed three times, resuspended, and 50 microliter drops of the sperm suspension were placed on 0.1% poly L-lysin pre-coated slide glasses (Erie Sci., Portsmouth, N.H.) for 20 min at 37 degrees Celsius. Sperm was incubated in 5% normal goat serum (GS) in dPBS for 3 hr at 4 degrees Celsius and labeled with anti-pPLC zeta (NT; 1:100) in 5% GS, overnight at 4 degrees Celsius. Samples were washed three times in 0.1% Tween 20 in dPBS (dPBS-T) and labeled with Alexa Fluor 555 goat anti-rabbit IgG (1:200) for 1 hr at RT. For acrosome staining, sperm were incubated with 20 micrograms/ml PNA-lectin (from Arachis hypogaea (peanut), Alexa Fluor 488 conjugated (Molecular Probes)) in dPBS for 30 min, at RT. After three washes with dPBS-T, samples were counterstained with 5 micrograms/ml Hoechst 33258 and mounted in VECTASHIELD mounting media (Vector Laboratories, UK). Fluorescence images were obtained using a Zeiss Axiovert 200M microscope with 63× oil immersion objective and Hamamatsu Orca AG cooled CCD Camera controlled through AxioVision software (Zeiss, Germany).

### Antibody Preparation

Anti-PLC zeta NT: rabbit serum was raised against a 19-mer sequence (MENKWFLSMVRDDFKGGKI) at the N-terminus of pig PLC zeta (accession no. BAC78817).

### EXPERIMENTS

### Example 1

### Validation of the Intracytoplasmic Injection Technique for Bovine Oocytes

Intracytoplasmic injections into bovine oocytes represent a challenge because of the high elasticity of the plasma membrane and the opacity and darkness of the bovine oocyte. In this study, we adapted the technique used for ICSI to inject consistent volumes of PLC zeta cRNA into bovine MII oocytes. Using this technique, we were able to inject a precise amount of media, confident of having penetrated the plasma membrane. To achieve this, a determined volume of media was loaded into a Fluorinert-filled pipette using a hydraulic microinjector. Then, the pipette was advanced into the oocyte up to about three-quarters of its diameter. By applying negative pressure, the oocyte cytoplasm was slowly aspirated. A well-defined meniscus was observed at the interface of the oocyte cytoplasm and the media when the plasma membrane was intact. When the plasma membrane was broken, the meniscus disappeared, and the flow of cytoplasm into the pipette was faster as a consequence of lower resistance. These two indicators were used to determine that the membrane had been trespassed. Then, applying positive pressure, the cytoplasm was injected back into the oocyte, followed by the media containing cRNA (FIG. 1a-c). The volume of media injected was controlled by observing the meniscus at the interface of media and Fluorinert, guided by the reticulum present in the microscope's field of view (FIG. 1d). According to our measurements of the internal diameter of the pipette and the length of the injected column of media, we calculated that the injection volume would be ˜6 pL. Measuring the diameter of oil drops released in aqueous media, we calculated an injection volume of 7±0.2 pL (range 6 to 8.2 pL) (FIG. 1e).

To corroborate the efficiency of the injection technique, we injected Texas Red dextran into the oocytes and then checked, under fluorescence excitation, the number of oocytes that had retained the dye. Out of 101 attempted injections, 99 resulted in successful injection of oocytes with clear red fluorescence in their cytoplasm (FIG. 1f). Subsequent activation of these oocytes—using ionomycin/DMAP—induced parthenogenetic development at similar rates to noninjected controls (data not shown). Moreover, the fluorescence intensity observed in the oocytes was similar among injected oocytes, indicating that the volume of media injected was consistent from oocyte to oocyte (FIG. 1f-i). Finally, parthenogenetically-activated sham injected oocytes developed at similar rates to noninjected controls—using ionomycin/DMAP—but did not develop when they remained untreated (FIG. 1j).

### Example 2

### Activation and Parthenogenetic Development of Bovine Oocytes Injected with PLC Zeta cRNA

We have previously shown that injection of mPLC zeta cRNA into bovine oocytes induces long-lasting [Ca²⁺]ᵢ oscillations (See Malcuit, C., et al., 2005. Fertilization and inositol 1,4,5-trisphosphate (IP3)-induced calcium release in type-1 inositol 1,4,5-trisphosphate receptor down-regulated bovine eggs. Biol Reprod. 73, 2-13). However, in those studies, we did not investigate the ability of mPLC zeta to induce oocyte activation or parthenogenetic development. In addition, whether or not injection of bPLC zeta cRNA could replicate the responses induced by bull sperm was not ascertained. To answer these pending questions, we first determined whether mPLC zeta cRNA was able to induce oocyte activation, which was monitored by the extrusion of the second polar body. When bovine oocytes were injected 22 hours after onset of maturation, extrusion of the second polar body was observed in 100 percent of the oocytes (n=13) within five hours of PLC zeta cRNA injection. Importantly, most of these oocytes cleaved to the two-cell stage and continued pre-implantation embryo development to the blastocyst stage at rates comparable to those observed in oocytes activated by the application of a common dual parthenogenetic procedure (ionomycin and DMAP; Table 1). We next examined whether injection of bPLC zeta cRNA was able to induce activation and embryo development of bovine oocytes. As shown in Table 2 (tenth dilutions), bPLC zeta effectively induced activation and embryo development to the blastocyst stage. We then investigated whether an association could be established between cRNA concentrations and high rates of pre-implantation embryo development. We first examined tenth dilutions of our cRNA stock and then refined the concentrations to obtain maximum embryo development. mPLC zeta cRNA was most effective when used at concentrations ranging from 0.25 to 1 μg/μL (Table 3). Remarkably, injection of bPLC zeta cRNAs was effective at inducing bovine oocyte activation and embryo development at concentrations nearly 5-fold lower than those required for mPLC zeta (Tables 2 and 3). Importantly, unlike mPLC zeta, the highest concentrations of bPLC zeta cRNA tested here had negative effects on both cleavage and blastocyst rates (Table 2).

With the optimal concentrations of m and bPLC zeta cRNAs determined, we investigated whether PLC zeta cRNAs induced pre-implantation embryo development to the blastocyst stage at rates comparable to those induced by IVF and by frequently used parthenogenetic procedures. Cleavage and blastocyst rates were similar among parthenogenetic embryos regardless of the activation method (FIG. 2a), although the cleavage and blastocyst rates of parthenogenetic embryos were higher than those of IVF-derived embryos (P<0.05). Notably, parthenogenetically activated zygotes consistently cleaved to the two-cell stage at earlier times than IVF embryos (FIG. 2b). Among parthenotes, a higher proportion of DMAP-activated embryos had cleaved by 18 hours postactivation, but no differences were observed thereafter (FIG. 2b). Collectively, our results show that injection of PLC zeta cRNAs induces high rates of pre-implantation bovine embryo development.

### Example 3

### Injection of PLC Zeta cRNAs Induces [Ca²⁺]ᵢ Oscillations in Bovine Oocytes

Our previous findings showing that concentration and PLC zeta species-of-origin affected the ability of PLC zeta cRNAs to induce embryo development suggested that the cRNAs under study were inducing specific [Ca²⁺]ᵢ responses, as it is well established that too low or excessive [Ca²⁺]ᵢ stimulation negatively impacts embryo development (See Ducibella, T., et al., 2002. Egg-to-embryo transition is driven by differential responses to Ca(2+) oscillation number. Dev Biol. 250, 280-91; See Gordo, A. C., et al., 2000. Injection of sperm cytosolic factor into mouse metaphase II oocytes induces different developmental fates according to the frequency of [Ca(2+)](i) oscillations and oocyte age. Biol Reprod. 62, 1370-9). Thus, we investigated the pattern of [Ca²⁺]ᵢ oscillations induced by the cRNA concentrations used to induce embryo development. Our results using mPLC zeta extend our previous findings (See Malcuit, C., et al., 2005. Fertilization and inositol 1,4,5-trisphosphate (IP3)-induced calcium release in type-1 inositol 1,4,5-trisphosphate receptor down-regulated bovine eggs. Biol Reprod. 73, 2-13) and show that sperm-like oscillations are induced over the first few hours of injection and then transition to a higher frequency of [Ca²⁺]ᵢ oscillations, which likely reflects protein accumulation with increased translation time (FIG. 3a). Regarding bPLC zeta, the lower cRNA concentration tested (0.1 μg/μL), which promoted high rates of embryo development, induced a pattern of oscillations similar to that induced by 1 μg/μL of mPLC zeta, and only transitioned to a high frequency of [Ca²⁺]ᵢ oscillations (less than three minute intervals) after 5 to 6 hours post-injection of the cRNA (FIG. 3b). Remarkably, 1 μg/μL of bPLC zeta cRNA induced [Ca²⁺]ᵢ oscillations that transitioned into high frequency oscillations by ˜3 hours, and in all oocytes the oscillations ceased completely by 6 hours after injection (FIG. 3) in all twelve evaluated oocytes. This complete cessation of [Ca²⁺]ᵢ oscillations was not observed during the timeframe analyzed in oocytes injected with 0.1 μg/μL bPLC zeta or with 1 μg/μL mPLC zeta cRNA.

### Example 4

### Injection of PLC Zeta cRNAs Induces IP₃R-1 Down Regulation in Bovine Oocytes

Fertilization-associated [Ca²⁺]ᵢ are underpinned by a steady production of IP₃ (See Jones, K. T., Nixon, V. L., 2000. Sperm-Induced Ca2+ Oscillations in Mouse Oocytes and Eggs Can Be Mimicked by Photolysis of Caged Inositol 1,4,5-Trisphosphate: Evidence to Support a Continuous Low Level Production of Inositol 1,4,5-Trisphosphate during Mammalian Fertilization. Developmental Biology. 225, 1-12.) that leads to down-regulation of IP₃R-1 (See Jellerette, T., et al., 2000. Down-regulation of the inositol 1,4,5-trisphosphate receptor in mouse eggs following fertilization or parthenogenetic activation. Dev Biol. 223, 238-50). Accordingly, we evaluated whether the oscillations induced by injection of PLC zeta cRNA resulted in IP₃R-1 degradation, and whether IP₃R-1 down regulation was associated with cRNA concentration and species-of-origin of the cRNAs. Oocytes were collected 12 hours after injection and mass of IP₃R-1 was evaluated by immunoblotting. We found that while injections of 0.1 μg/μL bPLC zeta or 1 μg/μL mPLC zeta cRNA induced fertilization-like loss of IP₃R-1 mass (FIG. 4), injection of 1 μg/μL bPLC zeta cRNA almost completely depleted IP₃R-1s from oocytes (FIG. 4). Together, these results confirm the notion that PLC zetas induce [Ca²⁺]ᵢ oscillations by stimulating IP₃ production and suggest that, while highly conserved, PLC zetas from different species have evolved to work more effectively in a species-specific manner.

### Example 5

### PLC Zeta cRNA-Activated Bovine Embryos Exhibit High Degree of Normal Chromosomal Composition

Activation of development in oocytes of large domestic species in the absence of fertilization requires the successive application of a Ca²⁺ ionophore followed by incubation for a few hours with a protein kinase or a protein synthesis inhibitor (See Alberio, R., et al., 2001. Mammalian oocyte activation: lessons from the sperm and implications for nuclear transfer. Int J Dev Biol. 45, 797-809). While these treatments have proven highly effective at inducing pre-implantation embryo development, they cause high rates of chromosomal abnormalities (See Bhak, J. S., et al., 2006. Developmental rate and ploidy of embryos produced by nuclear transfer with different activation treatments in cattle. Anim Reprod Sci. 92, 37-49; See Van De Velde, A., et al., 1999. Cell allocation and chromosomal complement of parthenogenetic and IVF bovine embryos. Mol Reprod Dev. 54, 57-62; See Winger, Q. A., et al., 1997. Bovine parthenogenesis is characterized by abnormal chromosomal complements: implications for maternal and paternal co-dependence during early bovine development. Dev Genet. 21, 160-6). Given we have shown that injection of PLC zeta cRNAs dose-dependently induce high rates of parthenogenetic pre-implantation development, we asked whether a higher proportion of these embryos showed normal chromosomal composition. Accordingly, we analyzed the chromosomal composition of eight-cell parthenogenetic embryos generated by injection of PLC zeta cRNAs versus that of embryos activated by two common chemical activation procedures, as well as IVF-derived embryos (Table 4; FIG. 5). Embryos generated using ionomycin/DMAP showed the highest proportion of abnormal ploidy (70%), while embryos activated using ionomycin/cycloheximide (33%) showed a modest amount of aneuploidy when compared to IVF-derived embryos (6%). PLC zeta cRNA-activated embryos exhibited the lowest percentage of aneuploid embryos among the parthenogenetic treatments, and the percentage, although higher (25%) was not significantly different to that observed in IVF-derived embryos. These results demonstrate that injection of PLC zeta cRNA is effective at inducing development of parthenogenetic embryos which exhibit normal chromosomal composition.

### Discussion

### PLC Zeta and Parthenogenetic Development

Our results show that a single injection of m or bPLC zeta cRNA into bovine oocytes induces high rates of oocyte activation and pre-implantation parthenogenetic development to the blastocyst stage. Notably, the rates of activation, embryonic cleavage, and development to the blastocyst stage were comparable to those observed after chemical activation methods. Unlike the latter procedures, where the [Ca²⁺]ᵢ rise induced by ionomycin is monotonic, is unable to persistently subdue MPF activity (See Kubiak, J. Z., et al., 1993. The metaphase II arrest in mouse oocytes is controlled through microtubule-dependent destruction of cyclin B in the presence of CSF. Embo J. 12, 3773-8) and requires the use of protein synthesis or protein kinase inhibitors to attain complete activation, injection of PLC zeta cRNA alone managed to induce all the events of oocyte activation. It is worth noting that the effectiveness of PLC zeta cRNA was dose-dependent, with either too low or too high of concentrations having detrimental effects on embryo development. Thus, while PLC zeta cRNA injection may require dose optimization according to the species under consideration, it may serve as an advantageous alternative to chemical activation protocols used to induce parthenogenetic and somatic cell nuclear transfer embryo development. Moreover, under the latter conditions, since the sperm [Ca²⁺]ᵢ pattern will be closely replicated, it would be possible to discern whether or not reprogramming defects commonly associated with nuclear transfer are at all due to aberrancies in [Ca²⁺]ᵢ signaling.

Embryo cleavage and development to blastocyst stage were higher in parthenotes than in IVF-derived embryos. The quality of oocytes utilized for these two procedures may explain these differences. For parthenogenetic activation, oocytes were denuded from the cumulus cells and only MII-stage oocytes (based on the presence of a polar body) were used. Moreover, denuding oocytes from the cumulus cells allowed for a stringent selection of good quality oocytes (with evenly granulated cytoplasm). In contrast, for IVF, less strict oocyte selection is performed, as the procedure utilizes cumulus-enclosed oocytes, resulting in insemination of a percentage of immature oocytes that have an inherently lower developmental potential.

Embryos derived from IVF cleaved on average six to twelve hours later than those of parthenogenetic origin. During IVF, fertilization takes place within a period of 6 hours after insemination (See Xu, K. P., Greve, T., 1988. A detailed analysis of early events during in-vitro fertilization of bovine follicular oocytes. J Reprod Fertil. 82, 127-34), while in the case of parthenotes, the time of activation is synchronized by ionomycin treatment or injection of PLC zeta cRNA. Parthenogenetic embryos activated by using a combination of ionomycin/DMAP started to cleave earlier than those activated by ionomycin/cycloheximide treatment. The basis for this difference remains unclear, although a more rapid decline in MPF and MAPK has been associated with this treatment (See Liu, L., Yang, X., 1999. Interplay of maturation-promoting factor and mitogen-activated protein kinase inactivation during metaphase-to-interphase transition of activated bovine oocytes. Biol Reprod. 61, 1-7).

Alterations of the chromosome composition in parthenogenetic bovine embryos have been frequently reported (See Bhak, J. S., et al., 2006. Developmental rate and ploidy of embryos produced by nuclear transfer with different activation treatments in cattle. Anim Reprod Sci. 92, 37-49; See Van De Velde, A., et al., 1999. Cell allocation and chromosomal complement of parthenogenetic and IVF bovine embryos. Mol Reprod Dev. 54, 57-62; See Winger, Q. A., et al., 1997. Bovine parthenogenesis is characterized by abnormal chromosomal complements: implications for maternal and paternal co-dependence during early bovine development. Dev Genet. 21, 160-6). Tetraploidy, the most common abnormality observed, may result from fusion of two blastomeres or from nuclear division without cytoplasmic division (See Hare, W. C., et al., 1980. Chromosomal analysis of 159 bovine embryos collected 12 to 18 days after estrus. Can J Genet Cytol. 22, 615-26). The basis for these abnormalities is not clear, but it is well established that the protocol used for oocyte activation affects the rate of aneuploidy (See Bhak, J. S., et al., 2006. Developmental rate and ploidy of embryos produced by nuclear transfer with different activation treatments in cattle. Anim Reprod Sci. 92, 37-49). The frequency of aneuploid embryos is greater when DMAP is used in the activation protocol than when cycloheximide is used (See Bhak, J. S., et al., 2006. Developmental rate and ploidy of embryos produced by nuclear transfer with different activation treatments in cattle. Anim Reprod Sci. 92, 37-49), and our study shows analogous results. Remarkably, injection of PLC zeta CRNA managed to activate bovine oocytes without causing a significant increase in the frequency of aneuploid embryos when compared to IVF-derived embryos. A quarter of PLC zeta-activated embryos were tetraploid, which is within the range of chromosomal abnormalities found in bovine IVF embryos (15 to 30 percent) (See Kawarsky, S. J., et al., 1996. Chromosomal abnormalities in bovine embryos and their influence on development. Biol Reprod. 54, 53-9; See Viuff, D., et al., 2002. Bovine embryos contain a higher proportion of polyploid cells in the trophectoderm than in the embryonic disc. Mol Reprod Dev. 62, 483-8). Importantly, the slight increase in tetraploids observed among PLC zeta cRNA-injected oocytes may be due to the use of cytochalasin, the application of which is required to obtain diploidization of parthenogenetically generated zygotes. Collectively, these results indicate that certain chemicals used for parthenogenetic activation may be responsible for the higher incidence of aneuploidy observed in bovine parthenotes and SCNT embryos, and these deficiencies of the prior art methods may be overcome by uses of PLC zeta as described herein.

We also report herein that injection of PLC zeta cRNAs into bovine oocytes induces long-lasting [Ca²⁺]ᵢ oscillations that are remarkably similar to those induced by the sperm in this species. The injection of the PLC zeta cRNAs was directly responsible for the [Ca²⁺]ᵢ responses, as [Ca²⁺]ᵢ oscillations were precluded when PLC zeta cRNA injection took place in the presence of cycloheximide, which inhibited cRNA translation. Likewise, [Ca²⁺]ᵢ oscillations were not observed when bovine oocytes were injected with PLCδ1 cRNA, a closely related family member (data not shown).

In bovine oocytes, fertilization-associated [Ca²⁺]ᵢ oscillations occur approximately every 20 minutes (See Nakada, K., et al., 1995. Initiation, persistance, and cessation of the series of intracellular Ca2+ responses during fertilization of bovine eggs. Journal of reproduction and development. 41, 77-84), although variable [Ca²⁺]ᵢ oscillations patterns have been observed, with one to five [Ca²⁺]ᵢ transients recorded during a 60-minute period (See Fissore, R. A., et al., 1992. Patterns of intracellular Ca2+ concentrations in fertilized bovine eggs. Biol Reprod. 47, 960-9), possibly due to highly variable oocyte quality. Injection of PLC zeta cRNA into bovine oocytes nearly replicates the sperm-induced [Ca²⁺]ᵢ oscillatory pattern, at least for the first hours after injection. Inevitably, and regardless of concentration and species of origin, the frequency of PLC zeta cRNA-induced oscillations increases, with oscillations occurring in less than three minute intervals. This change in the pattern of oscillations is likely to reflect protein accumulation as a result of translation of the cRNA and the time at which it occurs is influenced by cRNA concentration and species of origin. Under our conditions, high concentrations of bPLC zeta cRNAs had the most dramatic effect, with oscillations ceasing completely by six hours after injection. The underlying mechanism responsible for the termination of the oscillations is not yet clear, although the almost complete downregulation of IP₃R-1 observed in oocytes injected with 1 μg/μL of bPLC zeta cRNA might be a contributing factor (See Jellerette, T., et al., 2000. Down-regulation of the inositol 1,4,5-trisphosphate receptor in mouse eggs following fertilization or parthenogenetic activation. Dev Biol. 223, 238-50). Other factors, such as IP₃R-1 dephosphorylation (See Jellerette, T., et al., 2004. Cell cycle-coupled [Ca(2+)](i) oscillations in mouse zygotes and function of the inositol 1,4,5-trisphosphate receptor-1. Dev Biol. 274, 94-109; See Lee, B., et al., 2006a. Phosphorylation of IP3R1 and the regulation of [Ca2+]i responses at fertilization: a role for the MAP kinase pathway. Development. 133, 4355-65), or endoplasmic reticulum reorganization (See FitzHarris, G., et al., 2003. Cell cycle-dependent regulation of structure of endoplasmic reticulum and inositol 1,4,5-trisphosphate-induced Ca2+ release in mouse oocytes and embryos. Mol Biol Cell. 14, 288-301; See Fitzharris, G., et al., 2007. Changes in endoplasmic reticulum structure during mouse oocyte maturation are controlled by the cytoskeleton and cytoplasmic dynein. Dev Biol. 305:133-44) cannot be discounted.

It is interesting to note that cRNA concentrations that stimulated too low or too high [Ca²⁺]ᵢ of a response induced the lowest cleavage and developmental rates. These data are consistent with findings showing that the pattern of [Ca²⁺]ᵢ oscillations affects parthenogenetic oocyte activation events (See Ducibella, T., et al., 2002. Egg-to-embryo transition is driven by differential responses to Ca(2+) oscillation number. Dev Biol. 250, 280-91; See Ducibella, T., et al., 2006. Role of calcium signals in early development. Semin Cell Dev Biol. 17, 324-32; See Ozil, J. P., et al., 2005. Egg activation events are regulated by the duration of a sustained [Ca2+]cyt signal in the mouse. Dev Biol. 282, 39-54). For example, it was demonstrated that a different number of [Ca²⁺]ᵢ transients is required to initiate each event of oocyte activation and that a greater number of transients is needed to complete these events (See Ducibella, T., et al., 2002. Egg-to-embryo transition is driven by differential responses to Ca(2+) oscillation number. Dev Biol. 250, 280-91). It is reasonable to speculate that the shorter duration of the oscillatory [Ca²⁺]ᵢ transients we observed with the injection of 1 μg/μL of bPLC zeta was not sufficient to initiate and/or complete certain physiological functions required for the development of parthenogenetic embryos to the blastocyst stage. Alternatively, the overwhelming stimulation of the phosphoinositide pathway may have been detrimental to unknown cellular functions required for embryo development. Importantly, our observation that excessive [Ca²⁺]ᵢ oscillations induced by injection of PLC zeta cRNA results in lower cleavage rates and embryo development agrees with previous reports indicating that high concentrations of PLC zeta can inhibit development of parthenogenetic embryos (See Cox, L. J., et al., 2002. Sperm phospholipase Czeta from humans and cynomolgus monkeys triggers Ca2+ oscillations, activation and development of mouse oocytes. Reproduction. 124, 611-23; See Rogers, N. T., et al., 2004. Phospholipase Czeta causes Ca2+ oscillations and parthenogenetic activation of human oocytes. Reproduction. 128, 697-702).

The sperm factor is not species specific, as injection of sperm preparations from a variety of mammalian species were able to trigger fertilization-like [Ca²⁺]ᵢ oscillations in oocytes from different species (See Palermo, G. D., et al., 1997. Human sperm cytosolic factor triggers Ca2+ oscillations and overcomes activation failure of mammalian oocytes. Mol Hum Reprod. 3, 367-74; See Wu, H., et al., 1997. Injection of a porcine sperm factor triggers calcium oscillations in mouse oocytes and bovine eggs. Mol Reprod Dev. 46, 176-89; See Wu, H., et al., 1998. Injection of a porcine sperm factor induces activation of mouse eggs. Mol Reprod Dev. 49, 37-47). Consistent with this view, injection of PLC zeta cRNA coding for the human, simian, and mouse proteins induced [Ca²⁺]ᵢ oscillations and parthenogenetic development of oocytes from non-homologous species (See Cox, L. J., et al., 2002. Sperm phospholipase Czeta from humans and cynomolgus monkeys triggers Ca2+ oscillations, activation and development of mouse oocytes. Reproduction. 124, 611-23). Here we extend those results and show that mPLC zeta is very efficient at inducing [Ca²⁺]ᵢ oscillations and parthenogenetic development in bovine oocytes. Remarkably, bPLC zeta was much more active in bovine oocytes, as up to a 5-fold lower cRNA concentration was required to induce [Ca²⁺]ᵢ oscillations and activation responses comparable to those induced by mPLC zeta. Interestingly, the opposite response was observed when m and b PLC zeta cRNAs were injected in mouse oocytes (data not shown). While variations in the activity of PLC zetas from different species have been described elsewhere, with human PLC zeta being more effective at inducing [Ca²⁺]ᵢ oscillations in mouse oocytes than in simian or mouse PLC zeta (See Cox, L. J., et al., 2002. Sperm phospholipase Czeta from humans and cynomolgus monkeys triggers Ca2+ oscillations, activation and development of mouse oocytes. Reproduction. 124, 611-23), this is the first demonstration that a homologous form of the PLC zeta protein is more effective within species than across species. Therefore, while the mechanism of initiation of [Ca²⁺]ᵢ oscillations in mammals is highly conserved, adaptations have occurred during evolution to carefully orchestrate the early events of oocyte activation, the appropriate conclusion of which will impact embryo development to term.

We have presented herein that murine and bovine PLC zeta cRNA injection into bovine oocytes induces [Ca²⁺]ᵢ oscillations, IP₃R-1 downregulation and parthenogenetic development up to the blastocyst stage in a dose-dependent manner. Also, at optimal concentrations, PLC zeta cRNAs not only induce parthenogenetic development at rates comparable to those observed after using common chemical activation protocols, but also the generated embryos exhibit lower levels of aneuploidy. Hence, PLC zeta cRNAs could be used to parthenogenetically activate oocytes and to decipher the impact of [Ca²⁺]ᵢ oscillations on mammalian development.

### Example 6

### PLC Zeta Initiates Species-Specific [Ca2+]i Oscillations in Mammalian Eggs

PLC zeta has been found in all mammalian species examined to date. To determine whether PLC zeta derived from one species is effective activation of oocytes of another species, we injected varying concentrations of mouse and bovine PLC zeta into mouse and bovine oocytes and monitored the cells for Ca2+i oscillations. As expected, higher concentrations of PLZ zeta resulted in more frequent Ca2+i oscillations; however, at each concentration, murine PLC zeta elicited a lower frequency of Ca2+i oscillations in bovine cells than in murine cells (FIG. 6a). In contrast, bovine PLC zeta elicited a greater frequency of [Ca2+]i oscillations in bovine cells than in murine cells (FIG. 6b). These data indicate that PLC zeta initiates species-specific [Ca2+]i oscillations in mammalian eggs.

### Example 7

### Injection of Sperm from Low Fertility Patients into Mouse Eggs Initiates Highly Sifferent [Ca2+]i Responses

Introduction of human sperm into mouse eggs results in varying [Ca2+]i responses. To quantify the results and permit comparison of different donor groups (e.g. patients exhibiting low fertility and normal controls), [Ca2+]i oscillations observed after sperm injection may be divided into categories depending on the number of oscillations observed in one hour following injection. Traces representing different categories of [Ca2+]i oscillations observed after introduction of sperm from patients exhibiting low fertility or controls having normal fertility are shown (FIG. 7).

Significantly lower concentrations of PLC zeta cRNA were required to induce oscillations and activation of oocytes when the source of PLC zeta was the same species as the oocyte, than when the cRNA and oocyte were of different species, suggesting that while PLC zeta is highly conserved among mammals, adaptations have occurred that confer higher specific activity within species.

### Example 8

### [Ca2+]i Responses Elicited in Mouse Eggs by Human Sperm Vary According to Fertility Status

To determine defective induction of [Ca2+]i oscillations could be a cause of male infertility, we injected sperm from human patients exhibiting low fertility or controls having normal fertility into mouse oocytes and observed the resultant [Ca2+]i oscillations for one hour. To facilitate analysis, [Ca2+]i oscillations were divided into three categories: “<2” having two or fewer oscillations; “3˜9” having between three and nine oscillations (inclusive); and “>10” having ten or more oscillations.

The majority of sperm derived from the low fertility group elicited two or fewer [Ca2+]i oscillations in mouse oocytes (FIG. 8A), whereas the majority of sperm derived from the normal fertility group elicited ten or more [Ca2+]i oscillations. These results indicate that some cases of human infertility are caused by a failure to elicit [Ca2+]i oscillations.

### Example 9

### PLC Zeta is Present in the Equatorial Region of Control Subjects Having Normal Fertility

To determine the localization of PLC zeta in human sperm, we immunostained sperm with an antibody to PLC zeta peptide, previously described (See Knott, J. G., et al., 2005. Transgenic RNA interference reveals role for mouse sperm phospholipase Czeta in triggering Ca2+ oscillations during fertilization. Biol Reprod. 72, 992-6). We observed PLC zeta in an equatorial ring around the sperm head.

### Example 10

### PLC Zeta is Present in the Equatorial Region of Control Subjects Having Normal Fertility But is Absent from this Region in Patients of Low Fertility

We obtained samples of sperm from patients exhibiting male infertility and examined whether PLC zeta localization was altered in these individuals. For each of three patients, PLC zeta immunostaining was absent from the equatorial region, indicating that infertility likely results from insufficiency of PLC zeta.

### Example 11

### Additional Patients with Low Fertility Lack PLC Zeta the Equatorial Region of Sperm

We obtained samples of sperm from additional patients exhibiting male infertility and examined whether PLC zeta localization was altered in these individuals. For each of three patients, PLC zeta immunostaining was absent from the equatorial region (FIG. 11A), indicating that infertility likely results from insufficiency of PLC zeta, whereas each control subject exhibited the characteristic equatorial ring of PLC zeta staining (FIG. 11B).

### Example 12

### PLC Zeta has Species-Specific Localization in the Sperm Head

Immunostaining was used to detect PLC zeta localization. Bull sperm exhibit PLC zeta localized to an equatorial band (FIG. 12A). Mouse sperm exhibit PLC zeta localized to a hemispheric section plus a region covering the tip (FIG. 12B).

### Example 13

### Absence of PLC Zeta in Sperm of a Patient Having Male Infertility

One patient that had failed to produce a pregnancy after two attempts of ICSI (a total of 38 eggs) was examined for the presence of PLC zeta in sperm. The normal equatorial PLC zeta localization was undetectable by immunostaining of this patient's sperm (FIG. 13A). This patient's sperm looks abnormal in shape, although this patient had sperm that showed normal motility (data not shown). This patient's sperm also failed to induce [Ca2+]i oscillations when injected into mouse oocytes (FIG. 13B). PLC zeta protein was also undetectable in the patient's sperm by western blotting (FIG. 13C). These results indicate the failure to produce a pregnancy was caused by the absence of PLC zeta.

### Example 14

### ICSI Failure can be Rescued by Injection of PLC Zeta cRNA

For the patient of Example 13, further analysis was conducted to determine whether the defective oocyte activation could be rescued by introduction of exogenous PLC zeta. A human control subject having normal fertility was able to elicit [Ca2+]i oscillations when injected into mouse oocytes (FIG. 14A). In contrast, the patient exhibiting male infertility associated with undetectable PLC zeta in sperm did not elicit any [Ca2+i] oscillations when injected into mouse oocytes (FIG. 14B). However, when those same patient's sperm were injected into mouse oocytes together with mouse PLC zeta cRNA, [Ca2+]i oscillations were elicited similar to those observed with the control sperm (FIG. 15C). These experiment provides the first demonstration that patients lacking sperm PLC zeta can have normal [Ca2+]i oscillations restored upon fertilization through introduction of exogenous PLC zeta.

### Example 15

### PLCZ Triggers Fertilization-Like [Ca²⁺]ᵢ Oscillations in Bovine Oocyte Reconstructed by SCNT

As shown herein, injection of PLCZ cRNA into bovine oocytes induces long lasting [Ca²⁺]ᵢ oscillations, downregulation of the IP₃R-1 and supports parthenogenetic development to the blastocyst stage. In the following examples, we further extend these findings and demonstrate that PLCZ triggers fertilization-like [Ca²⁺]ᵢ oscillations in oocytes reconstructed by SCNT (FIG. 15). During the first 3 hours after injection a series of [Ca²⁺]ᵢ oscillations were observed with intervals of 28 minutes, and then their frequency increased to one oscillation every 8 minutes from 5 to 9 hours after PLCZ injection (Table 6).

Some oocytes (5/10) stopped oscillating during the recorded period at 9, 10.5, 11, 12, and 13 hours post injection of PLCZ. For those in which oscillations continued, the frequency started to decrease with oscillations occurring every 26 minutes from 11-14 hours post-activation. The initial pattern of oscillations (1-5 hours) was similar to that elicited by fertilization (See, Fissore R A, Dobrinsky J R, Balise J J, Duby R T, Robl J M. Patterns of intracellular Ca2+ concentrations in fertilized bovine eggs. Biol Reprod 1992; 47: 960-969; see, Nakada K, Mizuno J, Shiraishi K, Endo K, Miyazaki S. Initiation, persistance, and cessation of the series of intracellular Ca2+ responses during fertilization of bovine eggs. Journal of reproduction and development 1995; 41: 77-84); however, the increased frequency of [Ca²⁺]ᵢ oscillations in SCNT embryos between 5 and 9 hours after PLCZ injection is not observed after IVF and likely reflects protein accumulation with increased translation time. The frequency subsequently decreased to a fertilization-like rate, with oscillations lasting for more than 12 hours in most of the embryos.

The ability of PLCZ cRNA injection to induce repetitive [Ca²⁺]ᵢ rises provides a method to mimic the sperm-induced oocyte activation stimulus and to test the hypothesis that a more physiological oocyte activation stimulus can improve reprogramming after SCNT. Injection of boar sperm extract into bovine eggs also induces sperm-like [Ca²⁺]ᵢ oscillations and has been used to activate bovine SCNT embryos; however, the oscillations ceased 5 hours after the injection and IP₃R-1 downregulation was not observed (See, Knott J G, Poothapillai K, Wu H, He C L, Fissore R A, Robl J M. Porcine Sperm Factor Supports Activation and Development of Bovine Nuclear Transfer Embryos. Biol Reprod 2002; 66: 1095-1103). In mice, [Ca²⁺]ᵢ oscillations and oocyte activation are usually achieved by strontium chloride treatment, which is believed to act by sensitizing IP₃R to low IP₃ concentrations, causing the gating of IP₃R and Ca²⁺ release. In bovine oocytes however, strontium chloride does not induces [Ca²⁺]ᵢ oscillations (See, Malcuit C, Fissore R A. Activation of fertilized and nuclear transfer eggs. Adv Exp Med Biol 2007; 591: 117-131); moreover, in mice, strontium chloride does not downregulate IP₃R, suggesting that the PI pathway is not completely activated (See, Jellerette T, He C L, Wu H, Parys J B, Fissore R A. Down-regulation of the inositol 1,4,5-trisphosphate receptor in mouse eggs following fertilization or parthenogenetic activation. Dev Biol 2000; 223: 238-250).

Along with our observations that PLCZ cRNA induces [Ca²⁺]ᵢ oscillations, IP₃R-1 downregulation and parthenogenetic development when injected into MII oocytes, these data suggest that injection of PLCZ cRNA into oocytes reconstructed by SCNT can be used as an activation stimulus that mimics sperm-induced activation.

### Example 16

### Embryonic Development of Cloned Embryos Activated by PLCZ cRNA Injection

To evaluate whether PLCZ mRNA injection supports activation and in vitro development of bovine SCNT embryos, we injected 1 μg/μl of mPLCZ cRNA into oocytes reconstructed by nuclear transfer and compared cleavage and development rates induced by this treatment with those induced by chemical activation and by in vitro fertilization. Results are presented in Table 7 and representative figures of blastocysts obtained with the different activation protocols are shown in FIG. 16. A total of 769 bovine SCNT embryos were produced in 9 replicates. Equivalent cleavage rates were observed among SCNT groups, ranging from 75.4% to 79.2% (P>0.05). The cleavage rate of IVF (87.0%) embryos was higher than those of SCNT embryos (P<0.05). Development of the embryos to blastocyst stage was highest for SCNT embryos activated with Iono/CHX and lowest for embryos produced by IVF (P<0.05). Although statistically different, all groups produced blastocysts at rates (25-36%) comparable to those reported in the literature for IVF and SCNT embryos (See, Kane M T. A review of in vitro gamete maturation and embryo culture and potential impact on future animal biotechnology. Animal Reproduction Science 2003; 79: 171-190; see, Cibelli J B, Stice S L, Golueke P J, Kane J J, Jerry J, Blackwell C, Ponce de Leon F A, Robi J M. Cloned transgenic calves produced from nonquiescent fetal fibroblasts. Science 1998; 280: 1256-1258; see, Zakhartchenko V, Durcova-Hills G, Stojkovic M, Schernthaner W, Prelle K, Steinborn R, Muller M, Brem G, Wolf E. Effects of serum starvation and re-cloning on the efficiency of nuclear transfer using bovine fetal fibroblasts. J Reprod Fertil 1999; 115: 325-331; see, Heyman Y, Chavatte-Palmer P, LeBourhis D, Camous S, Vignon X, Renard J P. Frequency and occurrence of late-gestation losses from cattle cloned embryos. Biol Reprod 2002; 66: 6-13). Moreover, most of the generated blastocysts presented excellent morphological properties in all treatment groups, according to the international embryo transfer society (IETS) guidelines for evaluation of bovine embryos (See, Wright J M. Photographic illustrations of embryo developmental stage and quality codes. In: Stringfellow D A, Seidel S M (eds.), Manual of the International Embryo Transfer Society (third ed.). Savoy, Ill.: IETS; 1998: 167-170).

Total cell number and number of cells allocated to the TE and ICM have been regarded as valuable indicators of cattle embryo quality (See, van Soom A, Ysebaert M T, de Kruif A. Relationship between timing of development, morula morphology, and cell allocation to inner cell mass and trophectoderm in in vitro-produced bovine embryos. Mol Reprod Dev 1997; 47: 47-56; see, Van Soom A, Vanroose G, de Kruif A. Blastocyst evaluation by means of differential staining: a practical approach. Reprod Domest Anim 2001; 36: 29-35). A higher total cell number in the embryo correlated with developmental potential of IVF (See, van Soom A, Ysebaert M T, de Kruif A. Relationship between timing of development, morula morphology, and cell allocation to inner cell mass and trophectoderm in in vitro-produced bovine embryos. Mol Reprod Dev 1997; 47: 47-56) and SCNT embryos (See, Renard J P, Maruotti J, Jouneau A, Vignon X. Nuclear reprogramming and pluripotency of embryonic cells: Application to the isolation of embryonic stem cells in farm animals. Theriogenology 2007; 68 Suppl 1: S196-205). We determined the total number of cells and their allocation to inner cell mass (ICM) and trophectoderm (TE) of expanded/hatched blastocysts. No differences were observed in total, TE, or ICM cell numbers (FIG. 17); however, the ratio of ICM:TE cells was higher in SCNT embryos activated using DMAP than in those activated by using CHX or derived from IVF (P<0.05). PLCZ activated embryos did not differ from IVF or SCNT groups. It has been suggested that aberrant allocation of ICM and TE cells to the blastocyst stage may be responsible for the abnormalities observed after transfer of SCNT embryos (See, Koo D-B, Kang Y-K, Choi Y-H, Park J S, Kim H-N, Oh K B, Son D-S, Park H, Lee K-K, Han Y-M. Aberrant Allocations of Inner Cell Mass and Trophectoderm Cells in Bovine Nuclear Transfer Blastocysts. Biol Reprod 2002; 67: 487-492). Consistent with our observation, it has been reported that SCNT embryos activated using lonomycin/DMAP presented a significantly higher ratio of ICM:Total cells when compared to IVF and in vivo produced embryos (See, Koo D-B, Kang Y-K, Choi Y-H, Park J S, Kim H-N, Oh K B, Son D-S, Park H, Lee K-K, Han Y-M. Aberrant Allocations of Inner Cell Mass and Trophectoderm Cells in Bovine Nuclear Transfer Blastocysts. Biol Reprod 2002; 67: 487-492). We observed no differences between embryos activated using lonomycin/CHX or PLCZ cRNA injection and IVF controls, which could indicate that SCNT embryos activated by these means may have a better developmental potential.

We also determined the incidence of apoptosis among blastocysts produced with the different activation strategies. The proportion of apoptotic cells in blastocysts, as assessed by TUNEL staining, was similar among all the groups analyzed (P>0.05; FIG. 17). Finally, we performed chromosomal analysis at the blastocyst stage because maintenance of normal ploidy is a prerequisite for embryos to develop to term. The fibroblast donor cell line used in this study had a normal diploid chromosomal composition (FIG. 22), and after SCNT we observed that 86% of the embryos were diploid at the blastocyst stage irrespective of the activation protocol, and similar to IVF-derived embryos (FIG. 18; Table 8). This is in contrast to our observations of embryo ploidy after parthenogenetic activation. When embryos were parthenogenetically activated, the rate of aneuploidy was influenced by the activation treatment with a higher incidence of polyploidy in embryos activated using ionomycin/DMAP than with PLCZ cRNA injection. A similar difference between parthenogenetic and SCNT embryos has been previously reported (See, Bhak J S, Lee S L, Ock S A, Mohana Kumar B, Choe S Y, Rho G J. Developmental rate and ploidy of embryos produced by nuclear transfer with different activation treatments in cattle. Anim Reprod Sci 2006; 92: 37-49), while the basis for this remains unclear.

Taken together these results indicate that PLCZ mRNA injection is an effective method to induce activation of embryonic development after SCNT and that the embryos generated by this method present similar characteristics to embryos produced by IVF in terms of cell number and allocation, apoptosis and embryo ploidy.

### Example 17

### Effect of Oocyte Activation Method on Nuclear Reprogramming after Somatic Cell Nuclear Transfer

Reprogramming of gene expression involves reactivation of important embryonic genes as well as the repression of somatic cell-specific genes.

To test whether the type of activation stimulus has an effect on the reactivation of embryonic genes, we first evaluated the abundance of select transcripts at the 8-cell stage that have been shown to become transcriptionally active during embryonic genome activation. Expression levels in 8-cell embryos were normalized to an external control to account for differences in RNA extraction and RT efficiency.

Gene expression analysis at the 8-cell stage revealed that cloned embryos were able to express desmocollin 2 (DSC2) and glucose transporter 1 (GLUT1) at levels similar to IVF embryos (P>0.05; FIG. 19). Although, GLUT1 expression was affected by the type of SCNT activation protocol, with embryos activated using CHX presenting a higher level of transcripts than those activated by PLCZ cRNA injection (P<0.05). On the other hand, OCT4 (Oct-3, POU5F1) transcript abundance was significantly lower in DMAP-activated SCNT embryos compared to IVF derived embryos. Albeit OCT4 transcripts may be provided by the maternal pool supplied with the oocyte, OCT4 has been shown to be expressed at the 8-cell stage in bovine embryos. Thus, a lower level of OCT4 in lonomycin/DMAP activated-SCNT embryos could represent failure or incomplete initiation of OCT4 transcription.

We also performed gene expression analysis at the blastocyst stage to compare the level of reprogramming between the different activation stimuli. Blastocyst gene expression has been compared among IVF and SCNT embryos using RT-PCR (See, Daniels R, Hall V, Trounson A O. Analysis of gene transcription in bovine nuclear transfer embryos reconstructed with granulosa cell nuclei. Biol Reprod 2000; 63: 1034-1040; see, Daniels R, Hall V J, French A J, Korfiatis N A, Trounson A O. Comparison of gene transcription in cloned bovine embryos produced by different nuclear transfer techniques. Mol Reprod Dev 2001; 60: 281-288; see, Wrenzycki C, Wells D, Herrmann D, Miller A, Oliver J, Tervit R, Niemann H. Nuclear transfer protocol affects messenger RNA expression patterns in cloned bovine blastocysts. Biol Reprod 2001; 65: 309-317; see, Jang G, Jeon H Y, Ko K H, Park H J, Kang S K, Lee B C, Hwang W S. Developmental competence and gene expression in preimplantation bovine embryos derived from somatic cell nuclear transfer using different donor cells. Zygote 2005; 13: 187-195), quantitative real time PCR (See, Smith C, Berg D, Beaumont S, Standley N T, Wells D N, Pfeffer P L. Simultaneous gene quantitation of multiple genes in individual bovine nuclear transfer blastocysts. Reproduction 2007; 133: 231-242; see, Beyhan Z, Forsberg E J, Eilertsen K J, Kent-First M, First N L. Gene expression in bovine nuclear transfer embryos in relation to donor cell efficiency in producing live offspring. Mol Reprod Dev 2007; 74:18-27; see, de ACLS, Powell A M, do Vale Filho V R, Wall R J. Comparison of gene expression in individual preimplantation bovine embryos produced by in vitro fertilisation or somatic cell nuclear transfer. Reprod Fertil Dev 2005; 17: 487-496), and microarrays (See, Beyhan Z, Ross P J, Iager A E, Kocabas A M, Cunniff K, Rosa G J, Cibelli J B. Transcriptional reprogramming of somatic cell nuclei during preimplantation development of cloned bovine embryos. Dev Biol 2007; 305: 637-649; see, Pfister-Genskow M, Myers C, Childs L A, Lacson J C, Patterson T, Betthauser J M, Goueleke P J, Koppang R W, Lange G, Fisher P, Watt S R, Forsberg E J, Zheng Y, Leno G H, Schultz R M, Liu B, Chema C, Yang X, Hoeschele I, Eilertsen K J. Identification of differentially expressed genes in individual bovine preimplantation embryos produced by nuclear transfer: improper reprogramming of genes required for development. Biol Reprod 2005; 72: 546-555; see, Smith S L, Everts R E, Tian X C, Du F, Sung L Y, Rodriguez-Zas S L, Jeong B S, Renard J P, Lewin H A, Yang X. Global gene expression profiles reveal significant nuclear reprogramming by the blastocyst stage after cloning. Proc Natl Acad Sci USA 2005; 102: 17582-17587; see, Somers J, Smith C, Donnison M, Wells D N, Henderson H, McLeay L, Pfeffer P L. Gene expression profiling of individual bovine nuclear transfer blastocysts. Reproduction 2006; 131: 1073-1084; see, Wenli Zhou TXSWVFEHBFSSFARVAIP. Global gene expression analysis of bovine blastocysts produced by multiple methods. Molecular Reproduction and Development 2007); however, no clear picture of which genes are consistently misexpressed has yet emerged. This is not surprising considering that several aspects of the nuclear transfer procedure, including donor cell type, type of recipient cytoplast, enucleation and transfer procedures, activation method, and embryo culture environment diverge among the different studies. Some of these factors have been shown to affect gene expression of cloned embryos (See, Beyhan Z, Ross P J, lager A E, Kocabas A M, Cunniff K, Rosa G J, Cibelli J B. Transcriptional reprogramming of somatic cell nuclei during preimplantation development of cloned bovine embryos. Dev Biol 2007; 305: 637-649; see, Daniels R, Hall V J, French A J, Korfiatis N A, Trounson A O. Comparison of gene transcription in cloned bovine embryos produced by different nuclear transfer techniques. Mol Reprod Dev 2001; 60: 281-288; see, Wrenzycki C, Wells D, Herrmann D, Miller A, Oliver J, Tervit R, Niemann H. Nuclear transfer protocol affects messenger RNA expression patterns in cloned bovine blastocysts. Biol Reprod 2001; 65: 309-317; see, Jang G, Jeon H Y, Ko K H, Park H J, Kang S K, Lee B C, Hwang W S. Developmental competence and gene expression in preimplantation bovine embryos derived from somatic cell nuclear transfer using different donor cells. Zygote 2005; 13: 187-195, see, Beyhan Z, Forsberg E J, Eilertsen K J, Kent-First M, First N L. Gene expression in bovine nuclear transfer embryos in relation to donor cell efficiency in producing live offspring. Mol Reprod Dev 2007; 74:18-27; see, Wenli Zhou TXSWVFEHBFSSFARVAIP. Global gene expression analysis of bovine blastocysts produced by multiple methods. Molecular Reproduction and Development 2007).

We evaluated the expression of genes that are characteristic of the two cell lineages that comprise the blastocyst. OCT4, NANOG and SOX2 have been characterized as important for pluripotency and ICM formation, while the transcription factor CDX2 and the FGF receptor type 2 (FGFr2) have been shown to be expressed specifically in the TE of mouse embryos. We also determined the expression of TRYP8, a gene expressed by the somatic cell but not during preimplantation development of fertilized embryos, and of U2AF1L2, a gene found to discriminate between IVF and SCNT embryos in a previous study (See, Beyhan Z, Ross P J, Iager A E, Kocabas A M, Cunniff K, Rosa G J, Cibelli J B. Transcriptional reprogramming of somatic cell nuclei during preimplantation development of cloned bovine embryos. Dev Biol 2007). Blastocyst gene expression was normalized to the exogenous control and then to the total cell number in the embryo, as determined just before embryos lysis. We found that GAPDH, OCT4 and CDX2 were expressed at significantly lower levels in CHX activated SCNT embryos than in the other groups (FIG. 20).

It is interesting that the expression of a housekeeping gene (GAPDH), a transcription factor important for ICM development (OCT4), and a transcription factor important for TE development (CDX2) were expressed at lower levels in SCNT embryos activated by ionomycin/CHX compared to the other groups; however, blastocysts activated using lono/DMAP showed a pattern of gene expression similar to those activated by PLCZ. Thus, this difference can only be attributed to the use of CHX, given that the ionomycin and CB treatment are also included in lonomycin/DMAP and PLCZ activation protocols, respectively. Moreover, in agreement with our previous report (See, Beyhan Z, Ross P J, Iager A E, Kocabas A M, Cunniff K, Rosa G J, Cibelli J B. Transcriptional reprogramming of somatic cell nuclei during preimplantation development of cloned bovine embryos. Dev Biol 2007; 305: 637-649), U2AF1L2, a gene involved in RNA splicing, was only detected in IVF embryos, indicating that reprogramming of this locus failed in SCNT embryos irrespective of the activation protocol. Although the role of U2AF1L2 in embryonic development has not been investigated, the intrinsic capacity of this gene to affect several cellular processes could lead to potentially serious alterations in the ability of cloned embryos to develop normally. Finally, TRYP8, which was expressed at high levels in the donor cells, was amplified in a higher proportion (P<0.05) of SCNT embryos activated by CHX and DMAP (60% and 62.5%, respectively) than in IVF and SCNT embryos activated using PLCZ (11% and 33% respectively). Expression of donor cell-specific genes has been previously observed in cloned mice, suggesting that nuclear reprogramming may be incomplete after nuclear transfer (See, Gao S, Chung Y G, Williams J W, Riley J, Moley K, Latham K E. Somatic cell-like features of cloned mouse embryos prepared with cultured myoblast nuclei. Biol Reprod 2003; 69: 48-56). This epigenetic memory can have adverse consequence for embryonic development, as demonstrated by mouse cloning experiments in which nuclear transfer embryos developed better in donor cell culture medium than in embryo culture medium (See, Gao S, Chung Y G, Williams J W, Riley J, Moley K, Latham K E. Somatic cell-like features of cloned mouse embryos prepared with cultured myoblast nuclei. Biol Reprod 2003; 69: 48-56). Our finding that a more physiological activation stimulus (PLCZ) resulted in a reduction of somatic gene expression abnormalities indicates that oocyte activation plays a role in reprogramming the embryonic genome leading to erasure of somatic cell epigenetic memory. Similarly, it has been shown that a more physiological pattern of [Ca²⁺]ᵢ oscillations can affect gene expression at the blastocyst stage (See, Ozil J P, Banrezes B, Toth S, Pan H, Schultz R M. Ca2+ oscillatory pattern in fertilized mouse eggs affects gene expression and development to term. Dev Biol 2006; 300: 534-544) and that the Ca²⁺ signal is required at an appropriate level to induce translation of maternally stored mRNA in the early zygote (See, Ducibella T, Huneau D, Angelichio E, Xu Z, Schultz R M, Kopf G S, Fissore R, Madoux S, Ozil J P. Egg-to-embryo transition is driven by differential responses to Ca(2+) oscillation number. Dev Biol 2002; 250: 280-291), potentially affecting the reprogramming of the zygotic chromatin.

Our observation that GAPDH abundance was different between IVF embryos and SCNT embryos activated with ionomycin/CHX raises caution on the use of this gene as a control to normalize gene expression levels in studies comparing fertilized and cloned embryos. This is an interesting result given the fact that in several publications on gene expression analysis in embryos, GAPDH was the only reference gene used. If the employed reference gene fluctuates between samples, the subsequent normalization will produce erroneous results. In our study, the use of an external control gene added before RNA extraction accounts for variability in RNA extraction and RT efficiency. Nonetheless, it must be recognized that differences in sample quality or degradation during storage are not controlled by the exogenous control strategy. Also, the amount of sample material has to be carefully controlled for the external control to be effective. In the case of preimplantation embryos, the number of embryos allocated to each sample can be precisely determined; however, at the blastocyst stage, differences in embryo cell number can alter the initial total RNA input. Therefore, in our blastocyst samples we normalized gene expression levels to the total cell number of the embryos determined just before RNA extraction. The imaging methodology used for determining the number of cells in live embryos does not compromise the viability of mouse and bovine preimplantation embryos (See, Ross P J, Perez G I, Ko T, Yoo M S, Cibelli J B. Full developmental potential of mammalian preimplantation embryos is maintained after imaging using a spinning-disk confocal microscope. Biotechniques 2006; 41: 741-750). We have also determined that neither staining with Syto16 nor staining and imaging of the embryos affects the expression of GAPDH of bovine parthenogenetic embryos (FIG. 23). Coupled with the use of an external control, our method for cell number determination, although technically demanding, offers a reliable way to normalize gene expression levels, thus allowing the proper interpretation of blastocyst gene expression data.

Although the precise mechanism of nuclear reprogramming after SCNT has not been elucidated, it is known that chromatin remodeling plays a fundamental role. Chromatin remodeling involves changes in acetylation and methylation of histone tails, among other chromatin modifications. In the present study we evaluated genome-wide histone methylation at histone H3 lysine 27 (H3K27me3), associated with gene silencing, and histone acetylation at histone H4 lysine 5 (H4K5Ac), associated with transcriptional activation, by immunofluorescence (FIG. 21). We found that the levels of acetylated histone did not differ among groups; however, the levels of H3K27me3 were higher in bovine SCNT embryos activated using CHX or DMAP compared to those activated using PLCZ or derived from IVF (P<0.05; FIG. 21). Tri-methylation of histone H3 at lysine 27 is catalyzed by polycomb group complexes and is associated with stable and heritable gene silencing (See, Schuettengruber B, Chourrout D, Vervoort M, Leblanc B, Cavalli G. Genome regulation by polycomb and trithorax proteins. Cell 2007; 128: 735-745). We have previously analyzed the levels of H3K27me3 through preimplantation development of bovine IVF, parthenogenetic, and chemically activated SCNT embryos, and found that the levels of H3K27me3 decreased from the PN stage, reaching a minimum at the 8-cell stage, and then increasing at the blastocyst stage in all groups (FIGS. 24 and 25). In SCNT embryos activated by ionomycin/CHX the levels reached at the blastocyst stage were significantly higher than those attained by IVF and parthenogenetically derived embryos. Also, donor somatic cell nuclei were stained for H3K27me3 at higher levels than embryonic nuclei (FIG. 25). In the present study, we found that the levels of H3K27me3 were similar to IVF embryos when SCNT embryos were activated by a sperm-like stimulus (PLCZ), but higher when we used chemical activation. This observation is in agreement with our gene expression data where chemically activated embryos showed higher levels of somatic gene expression, suggesting that embryos activated by chemical means may retain a somatic-like pattern of epigenetic arrangement compared to PLC activated and fertilized embryos. The mechanism by which the activation system influences the reprogramming of H3K27me3 represents an interesting area for future research. Moreover, given the importance of H3K27me3 in conferring stem cell identity to embryonic stem cells (See, Azuara V, Perry P, Sauer S, Spivakov M, Jorgensen H F, John R M, Gouti M, Casanova M, Warnes G, Merkenschlager M, Fisher A G. Chromatin signatures of pluripotent cell lines. Nat Cell Biol 2006; 8: 532-538; see, Boyer L A, Plath K, Zeitlinger J, Brambrink T, Medeiros L A, Lee T I, Levine S S, Wernig M, Tajonar A, Ray M K, Bell G W, Otte A P, Vidal M, Gifford D K, Young R A, Jaenisch R. Polycomb complexes repress developmental regulators in murine embryonic stem cells. Nature 2006; 441: 349-353; Lee T I, Jenner R G, Boyer L A, Guenther M G, Levine S S, Kumar R M, Chevalier B, Johnstone S E, Cole M F, Isono K, Koseki H, Fuchikami T, Abe K, Murray H L, Zucker J P, Yuan B, Bell G W, Herbolsheimer E, Hannett N M, Sun K, Odom D T, Otte A P, Volkert T L, Bartel D P, Melton D A, Gifford D K, Jaenisch R, Young R A. Control of developmental regulators by Polycomb in human embryonic stem cells. Cell 2006; 125: 301-313), it is tempting to speculate that aberrant H3K27me3 in cloned embryos activated by chemical means may lead to abnormal cell differentiation thus resulting in developmental abnormalities and embryonic lethality.

In summary, we have utilized an oocyte activation treatment (PLCZ cRNA injection) that recapitulates the calcium oscillation patterns observed after fertilization. We demonstrate that this treatment supports activation and in vitro development of bovine nuclear transfer embryos at rates comparable to those induced by chemical activation and by fertilization. Also, the embryos activated using this protocol had similar characteristics in terms of cell number, ploidy, apoptosis, and chromatin modifications to in vitro fertilized embryos. Conversely, U2AF1L1 expression was not detected in any of the SCNT groups, indicating that some abnormalities common to SCNT embryos persist in clones activated by PLCZ cRNA injection; however, most gene expression alterations observed in chemically activated embryos were not evident in embryos activated by PLCZ cRNA injection.

TABLE 1
Injection of mouse PLC zeta cRNA in bovine eggs induces
parthenogenetic activation.

| | Ionomycin/DMAP | mPLC zeta |
|---|---|---|
| Oocytes (n) | 161 | 123 |
| Cleavage (%) | 81 | 88 |
| Blastocyst rate (%) | 18 | 23 |
| Blastocyst cell number | 63 ± 16 | 75 ± 17 |

TABLE 2
Optimization of mouse PLC zeta cRNA concentration
to activate bovine oocytes

| | mPLC zeta | | | | | |
|---|---|---|---|---|---|---|
| | cRNA | | Repli- | Oocytes | Cleavage | Blastocyst |
| Experiment | concentration | | cates | injected | rate | Rate |
| Tenth | 0.5 | μg/μL | 3 | 123 | 90.2^{a} | 32.5^{a} |
| dilutions | 0.05 | μg/μL | 3 | 112 | 83.9^{b} | 8.9^{b} |
| | 0.005 | μg/μL | 3 | 122 | 48.4^{c} | 0.8^{c} |
| Fine | 1 | μg/μL | 3 | 107 | 79.4^{a} | 33.6^{a} |
| tuning | 0.5 | μg/μL | 3 | 112 | 92.0^{a} | 33.9^{a} |
| | 0.25 | μg/μL | 3 | 110 | 85.5^{a} | 33.6^{a} |

^{a,b,c}Different superscripts indicate P < 0.05 (Chi square).

TABLE 3
Optimization of bovine PLC zeta cRNA concentration
to activate bovine oocytes

| | bPLC zeta | | | | | |
|---|---|---|---|---|---|---|
| | cRNA | | Repli- | Oocytes | Cleavage | Blastocyst |
| Experiment | concentration | | cates | injected | rate | Rate |
| Tenth | 1 | μg/μL | 3 | 110 | 49.1^{a} | 12.7^{a} |
| dilutions | 0.1 | μg/μL | 3 | 118 | 87.3^{b} | 29.7^{b} |
| | 0.01 | μg/μL | 3 | 112 | 69.6^{a} | 15.2^{a} |
| Fine | 0.5 | μg/μL | 4 | 144 | 70.8^{a} | 18.8^{a} |
| tuning | 0.1 | μg/μL | 4 | 154 | 88.3^{ab} | 29.9^{a} |
| | 0.05 | μg/μL | 4 | 145 | 89.0^{b} | 22.8^{a} |

^{a,b}Different superscripts indicate P < 0.05 (Chi square).

TABLE 4
Chromosomal composition of 8-cell embryos activated by different means

| | Embryos | Informative | | | | | | Mixoploid | | Total | |
|---|---|---|---|---|---|---|---|---|---|---|---|
| | Evaluated | embryos | 2 n | | 3 n | 4 n | | (2 n/4 n) | Other | Abnormal | |
| IVF | 22 | 17 | 16 | (94%) | 0 | 1 | (6%) | 0 | 0 | 1 | (6%)^{a} |
| mPLCζ | 28 | 16 | 12 | (75%) | 0 | 4 | (25%) | 0 | 0 | 4 | (25%)^{ab} |
| Iono/DMAP | 26 | 20 | 6 | (30%) | 0 | 10 | (50%) | 2 (10%) | 2 (10%) | 14 | (70%)^{c} |
| Iono/CHX | 28 | 18 | 12 | (67%) | 1 (5%) | 5 | (26%) | 0 | 1 | 6 | (33%)^{b} |

| | | |
|---|---|---|
| Total | 104 | 71 |

^{a,b,c}Different superscripts indicate P < 0.05 (Chi square).
A total of 167 metaphases were evaluated (2.3 per informative embryo).

TABLE 5
Primers used for quantitative real time RT-PCR.

| | GenBank | | Amplicon |
|---|---|---|---|
| Gene | accession number | Primer sequence | size |
| HcRed | | F: 5′-GCCCGGCTTCCACTTCA-3′ | 79 bp |
| | | R: 5′-GGCCTCGTACAGCTCGAAGTA-3′ | |
| CDX2 | XM_871005 | F: 5′-GCAAAGGAAAGGAAAATCAACAA-3′ | 84 bp |
| | | R: 5′-GGGCTCTGGGACGCTTCT-3′ | |
| DSC2 | XM_615164 | F: 5′-TGTTGCAGCGAACGACAAG-3′ | 75 bp |
| | | F: 5′-CCGCAAGTGTCCTAAATTTGG-3′ | |
| FGFr2 | XM_880481 | R: 5′-CTGGCAGCTAAATCTCGATGAA-3′ | 86 bp |
| | | R: 5′-GACCTGGTGTCGTGTACCTACCA-3′ | |
| GAPDH | BG691477 | F: 5′-GCCATCAATGACCCCTTCAT-3′ | 70 bp |
| | | R: 5′-TGCCGTGGGTGGAATCA-3′ | |
| GLUT1 | NM_174602 | F: 5′-TCCGGCAGGGAGGAGCAAGT-3′ | 177 bp |
| | | R: 5′-TGCTGAGATCTATCAGTTTGAG-3′ | |
| NANOG | DQ069776 | F: 5′-CGTGTCCTTGCAAACGTCAT-3′ | 66 bp |
| | | R: 5′-CTGTCTCTCCTCTTCCCTCCTC-3′ | |
| OCT4 | NM_174580 | F: 5′-CCACCCTGCAGCAAATTAGC-3′ | 68 bp |
| | | R: 5′-CCACACTCGGACCACGTCTT-3′ | |
| SOX2 | NM_001105463 | F: 5′-GGTTGACATCGTTGGTAATTTATAATAGC-3′ | 88 bp |
| | | R: 5′-CACAGTAATTTCATGTTGGTTTTTCA-3′ | |
| TRYP8 | NM_174690 | F: 5′-CCACACTCGGACCACGTCTT-3′ | 83 bp |
| | | R: 5′-AGCCAGCGCAGATCATGTT-3′ | |
| U2AF1L2 | XR_028361 | F: 5′-GGAGTAGTCATGAGGGCGAGAA-3′ | 78 bp |
| | | R: 5′-TTCCGCTGCTTTGAGAACTGT-3′ | |

HcRed Primers: See, Bettegowda A, Patel O V, Ireland J J, Smith G W. Quantitative analysis of messenger RNA abundance for ribosomal protein L-15, cyclophilin-A, phosphoglycerokinase, beta-glucuronidase, glyceraldehyde 3-phosphate dehydrogenase, beta-actin, and histone H2A during bovine oocyte maturation and early embryogenesis in vitro. Mol Reprod Dev 2006; 73: 267-278.

GAPDH Primers: See Bettegowda A, Patel O V, Ireland J J, Smith G W. Quantitative analysis of messenger RNA abundance for ribosomal protein L-15, cyclophilin-A, phosphoglycerokinase, beta-glucuronidase, glyceraldehyde 3-phosphate dehydrogenase, beta-actin, and histone H2A during bovine oocyte maturation and early embryogenesis in vitro. Mol Reprod Dev 2006; 73: 267-278.

U2AF1L2 Primers: See, Beyhan Z, Ross P J, Iager A E, Kocabas A M, Cunniff K, Rosa G J, Cibelli J B. Transcriptional reprogramming of somatic cell nuclei during preimplantation development of cloned bovine embryos. Dev Biol 2007; 305: 637-649)

TABLE 6
Time interval between [Ca²⁺]ᵢ oscillations in SCNT
embryos activated using PLCZ cRNA injection.

| | | Mean ± SEM interval |
|---|---|---|
| Time after | Oscillating | between [Ca²⁺]ᵢ |
| PLCZ injection | oocytes (n) | increases (min) |
| 1 to 3 h: | 10/10 | 28.4 ± 1.6 |
| 3 to 5 h: | 10/10 | 13.2 ± 2.6 |
| 5 to 7 h: | 10/10 | 8.1 ± 1.3 |
| 7 to 9 h: | 10/10 | 8.1 ± 2.1 |
| 9 to 11 h: | 9/10 | 19.8 ± 5.3 |
| 11 to 14 h: | 5/10 | 25.7 ± 6.3 |

TABLE 7
Preimplantation development of IVF and SCNT derived
embryos activated using different protocols.

| | Embryos | | |
|---|---|---|---|
| Treatment | cultured | Cleaved (%) | Blastocysts (%) |
| IVF | 492 | 428 (87.0)^{a} | 125 (25.4)^{a} |
| PLCZ | 332 | 262 (78.9)^{b} | 92 (27.7)^{ab} |
| Iono/DMAP | 327 | 259 (79.2)^{b} | 109 (33.3)^{bc} |
| Iono/CHX | 329 | 248 (75.4)^{b} | 118 (35.9)^{c} |

^{abc}Different superscripts indicate P < 0.05.

TABLE 8
Chromosomal composition of blastocysts activated by using different
protocols

| Treatment | n | Diploid | Tetraploid | Mixoploid | Total abnormal |
|---|---|---|---|---|---|
| IVF | 15 | 13 | 2 | | 2 (13.3%) |
| PLCZ | 12 | 10 | 1 | 1* | 2 (16.7%) |
| Iono/DMAP | 12 | 11 | 1 | | 1 (8.3%) |
| Iono/CHX | 20 | 17 | 2 | 1# | 3 (15.0%) |

*diploid/triploid;
#diploid/tetraploid

We claim:

1. A method of inducing or enhancing the activation or development potential of a first cell and/or its capability to develop into a zygote comprising:
(i) introducing into or contacting a first cell with an amount of phospholipase C zeta protein or a fragment or variant thereof or a nucleic acid sequence encoding any of the foregoing, or inducing said first cell to express an amount of phospholipase C zeta protein or a fragment or variant thereof, sufficient to induce or enhance the activation of said first cell and render said first cell capable of developing into a zygote upon activation by said phospholipase C zeta protein alone or in combination with another activating agent.

2. The method of claim 1 wherein the cell is contacted or injected with a nucleic acid sequence that results in the expression of said PLC zeta protein or fragment or variant.

3. The method of claim 2 wherein said nucleic acid sequence is selected from a cRNA, cDNA or vector containing.

4. The method of claim 1 wherein the cell is contacted or injected with a PLC zeta protein or functional fragment or variant.

5. The method of claim 1, wherein said first cell is selected from the group comprising:
a fertilized or unfertilized oocyte;
a nuclear transfer cell;
a parthenogenic embryonic cell;
an androgenetic embryonic cell;
a sperm; and
a somatic cell into which the cytoplasm of a second cell has been introduced.

6. The method of claim 5, wherein said first cell is an unfertilized oocyte.

7. The method of claim 6 wherein said first cell is an unfertilized human or bovine oocyte.

8. The method of claim 7, further comprising:
(ii) in vitro fertilization of said oocyte.

9. The method of claim 8, wherein said in vitro fertilization comprises injection or fusion of a sperm or DNA derived therefrom with said oocyte.

10. The method of claim 9, further comprising:
(iii) introducing said first cell into a host or culturing said activated, fertilized oocyte in vitro, whereby said cell develops into an embryo.

11. The method of claim 5, wherein said first cell is a nuclear transfer cell.

12. The method of claim 11 wherein said nuclear transfer cell is a cross-species nuclear transfer cell.

13. The method of claim 5 wherein said first cell is a somatic cell into which the cytoplasm of a second cell has been introduced.

14. The method of claim 13, wherein said second cell is an oocyte, primordial germ cell, inner cell mass cell or an embryonic stem cell.

15. The method of claim 14, wherein said somatic cell into which the cytoplasm of a second cell has been introduced is the product of fusion or insertion of a somatic cell with said second cell.

16. The method of claim 15, wherein the nucleus of said second cell is removed or destroyed before, synchronous or after introduction of said cytoplasm of said second cell.

17. The method of claim 14, wherein said somatic cell into which the cytoplasm of a second cell has been introduced is produced by injection or fusion of a somatic cell with cytoplasm or a cytoplast derived from said second cell.

18. The method of claim 5, wherein said phospholipase C zeta protein or a fragment or variant thereof comprises the cytoplasm of a third cell that is introduced into said first cell by injection or by cell-cell fusion.

19. The method of claim 18, wherein said third cell is engineered to express a gene encoding phospholipase C zeta or a fragment or variant thereof.

20. The method of claim 19, wherein said gene encoding phospholipase C zeta or a fragment or variant thereof is under the control of an inducible promoter.

21. The method of claim 19, wherein said gene encoding phospholipase C zeta or a fragment or variant thereof is coupled to a dominant selectable marker that results in the amplification of said phospholipase C zeta gene under specific selection conditions.

22. The method of claim 19 wherein the origin of said third cell is selected from ovine, bovine, caprine, primate, human, equine, murine, and leporine.

23. The method of claim 1 wherein said other activating agent is selected from the group consisting of a sperm, an unfertilized oocyte, or chromosomal DNA derived therefrom.

24. The method of claim 23 wherein the other activating agent comprises human sperm.

25. A method of detecting male infertility comprising:
(i) determining a patient's phospholipase C zeta level by measuring the level of phospholipase C zeta in a sperm sample from the patient;
(ii) comparing said patient's phospholipase C zeta level with a reference value, wherein said reference value is the minimum level of phospholipase C zeta observed in essentially all sperm samples from one or more control subjects known to exhibit normal fertility;
(iii) identifying the patient as having male infertility if said patient's phospholipase C zeta level is significantly lower than the reference value;
wherein said sperm sample from the patient and said sperm samples from one or more control subjects comprise sperm cells or sperm progenitor cells.

26. The method of claim 25, wherein the level of phospholipase C zeta in a sperm sample is the level of phospholipase C zeta protein in said sperm sample.

27. The method of claim 25, wherein the level of phospholipase C zeta in a sperm sample is the level of phospholipase C zeta mRNA in said sperm sample.

28. The method of claim 25, wherein said patient's phospholipase C zeta level is significantly lower than the reference value if said patient's phospholipase C zeta level that is less than about 90% of the reference value.

29. The method of claim 28, wherein said patient's phospholipase C zeta level is significantly lower than the reference value if said patient's phospholipase C zeta level that is less than about 50% of the reference value.

30. The method of claim 29, wherein said patient's phospholipase C zeta level is significantly lower than the reference value if said patient's phospholipase C zeta level that is less than about 10% of the reference value.

31. The method of claim 25 wherein said patient's phospholipase C zeta level is significantly lower than the reference value if said patient's phospholipase C zeta level that is no more than the phospholipase C zeta level of a negative control patient who has been found to exhibit male infertility associated with reduced levels of phospholipase C zeta.

32. A method of detecting male infertility comprising:
(i) determining the reference spatial localization of phospholipase C zeta protein, wherein said reference spatial localization of phospholipase C zeta protein is the spatial localization of phospholipase C zeta protein observed in the majority of sperm from the majority of control subjects known to have normal fertility, wherein said control subjects known to have normal fertility are of the same species as said patient;
(ii) determining fraction of patient's sperm showing spatial localization of phospholipase C zeta protein comparable to the reference spatial localization of phospholipase C zeta protein; and
(iii) identifying the patient as having male infertility if said fraction of patient's sperm showing spatial localization of phospholipase C zeta protein comparable to the reference spatial localization of phospholipase C zeta protein is significantly lower than 100%.

33. The method of claim 32, wherein said patient is identified as having male infertility if less than about 90% of said patient's sperm exhibit spatial localization of phospholipase C zeta protein comparable to the reference spatial localization of phospholipase C zeta protein.

34. The method of claim 33, wherein said patient is identified as having male infertility if less than about 50% of said patient's sperm exhibit spatial localization of phospholipase C zeta protein comparable to the reference spatial localization of phospholipase C zeta protein.

35. The method of claim 34, wherein said patient is identified as having male infertility if less than about 10% of said patient's sperm exhibit spatial localization of phospholipase C zeta protein comparable to the reference spatial localization of phospholipase C zeta protein.

36. The method of claim 35, wherein said patient is identified as having male infertility if less than about 1% of said patient's sperm exhibit spatial localization of phospholipase C zeta protein comparable to the reference spatial localization of phospholipase C zeta protein.

37. The method of claim 36, wherein said patient is identified as having male infertility if the fraction of said patient's sperm that exhibit spatial localization of phospholipase C zeta protein comparable to the reference spatial localization of phospholipase C zeta protein is similar to or lower than the fraction of sperm that exhibit spatial localization of phospholipase C zeta protein comparable to the reference spatial localization of phospholipase C zeta protein that is observed in a negative control patient who has been found to exhibit male infertility associated with a reduced fraction of sperm exhibiting normal localization of phospholipase C zeta.

38. A method of treating male infertility, comprising:
(i) contacting an oocyte in vitro with sperm from a patient suspected or known to have male infertility;
(ii) introducing into or contacting said oocyte and/or sperm with a compatible phospholipase C zeta protein or a functional variant or fragment thereof or a nucleic acid sequence encoding any of the foregoing, or inducing said oocyte or sperm to express a phospholipase C zeta protein or a fragment or variant thereof; wherein steps (i) and (ii) may be effected in either order or simultaneous and result in an activated fertilized oocyte; and
(iii) thereafter, implanting said activated, fertilized oocyte in a suitable host or culturing said activated, fertilized oocyte in vitro, whereby said oocyte develops into an embryo.

39. A method of detecting male infertility, comprising:
(i) identifying non-functional allelic variants of phospholipase C zeta;
(ii) determining partial or complete genomic sequences of a patient's phospholipase C zeta genes; and
(iii) identifying the patient as having male infertility if said partial or complete genomic sequences of a patient's phospholipase C zeta genes are identical or similar to said non-functional allelic variants of phospholipase C zeta, or if said genomic sequences of a patient's phospholipase C zeta genes encode allelic variants of phospholipase C zeta having mutations that encode truncated phospholipase C zeta protein, or possess mutations within splice donor or splice acceptor sequences, or encode mutations within catalytic residues, or any combination thereof.

40. A composition for treatment of male infertility, comprising:
(i) a patient's sperm; and
(ii) an amount of phospholipase C zeta protein or a fragment or variant thereof, or an amount of nucleic acid encoding phospholipase C zeta or a fragment or variant thereof, sufficient to activate an oocyte and render said oocyte cell capable of developing into a zygote, upon injection of an amount of said composition into an oocyte.

41. A method of identifying a composition useful for treatment of male infertility, comprising:
(i) contacting one or more cells with a candidate composition;
(ii) monitoring the intracellular calcium concentration of said one or more cells; and
(iii) thereafter, identifying a composition as useful for treatment of male infertility if fluctuations of said intracellular calcium concentration are observed that are similar in frequency and amplitude to the fluctuations of intracellular calcium concentration observed when cells of the same type as said one or more cells are contacted with phospholipase C zeta or a nucleic acid sequence encoding.
